(19) United States
(12) Patent Application Publication
(10) Pub. No.: US 2025/0326884 A1
(43) Pub. Date: Oct. 23, 2025

(54) COMPOSITIONS COMPRISING AMINO ACID ADDUCTS AND HYDROXY SILANES, CONTROLLED RELEASE PARTICLES AND COMPOSITIONS COMPRISING SAME, AND METHODS OF MAKING SAME

(71) Applicant: TRUCAPSOL LLC, Bethlehem, PA (US)

(72) Inventors: Monoj PRAMANIK, Allentown, PA (US); Jiten DIHORA, Center Valley, PA (US)

(21) Appl. No.: 18/643,634

(22) Filed: Apr. 23, 2024

(51) Int. Cl. C08G 18/58; A01N 25/10; A01N 33/18; A01P 7/04; A61K 8/02; A61K 8/87; A61K 9/16; A61Q 13/00; A61Q 19/00; B01J 13/16; B01J 13/20; C08G 18/10; C08G 18/79; C11D 3/00; C11D 3/395; C11D 3/50; C11D 17/00; C11D 17/04

(52) U.S. Cl. CPC C08G 18/588; A01N 25/10; A01N 33/18; A01P 7/04; A61K 8/0233; A61K 8/87; A61K 9/1641; A61Q 13/00; A61Q 19/00; B01J 13/16; B01J 13/206; C08G 18/10; C08G 18/584; C08G 18/792; C11D 3/0015; C11D 3/3951; C11D 3/505; C11D 17/0039; C11D 17/043

(57) ABSTRACT

A composition includes at least one of: (a) an amino acid isocyanate adduct of Formula I:

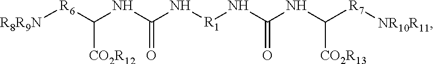

and salts thereof, and (b) an amino acid epoxide adduct of Formula II:

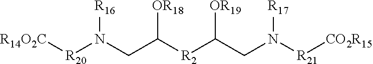

and salts thereof. R₁ is a moiety of a monomer or prepolymer including two —NCO functional groups, which have reacted to form urea linkages; R₂ is a moiety of a monomer or prepolymer including two glycidyl ether epoxide functional groups, which have reacted to form amino alcohol groups; R₆, R₇, R₂₀ and R₂₁ are independently (CH₂)ₙ, n is 1-6; R₈, R₉, R₁₀ and R₁₁ are independently hydrogen or CH₂X; R₁₂, R₁₃, R₁₄ and R₁₅ are independently hydrogen, alkali metal cation, or X; R₁₆ and R₁₇ are independently hydrogen or X; R₁₈ and R₁₉ are independently hydrogen, SiO₃ or X; and each X is independently hydrogen or a substituent. Controlled release particles, consumer products and methods including the composition are also disclosed.

### BACKGROUND OF THE INVENTION

### 1. Field of Invention

This invention relates to controlled release compositions, encapsulant compositions and methods for making and using them.

### 2. Description of Related Art

There are many microencapsulated delivery systems disclosed in the art to control the release of the encapsulated active or provide release when a specific trigger is applied. Such systems have previously suffered from several drawbacks.

Controlled release microcapsules available in dry powder form that provide release of active upon application of shear or friction generally suffer from several drawbacks: (1) such microcapsules are made of highly crosslinked membranes that make the capsules brittle and results in significant fracture of particles during the drying process, (2) despite such highly crosslinked membranes, the materials of construction of the membrane impart high permeabilities when incorporated into products that contain high levels of surfactant, solvents, and/or water, which results in the premature benefit agent release, (3) they can only effectively encapsulate a limited breadth of benefit agents, (4) they either are so stable that they do not release the benefit agent in use or have insufficient mechanical stability to withstand the processes required to incorporate them in and/or make a consumer product, and (5) they do not redisperse well when formulated into aqueous formulations.

In general, conventional microcapsules have a structure of an encapsulated active composition enclosed in a polymeric shell. The microcapsules have a polymeric shell which is generally obtained by any one of: (a) condensation reactions, (b) free radical polymerization reactions, (c) interfacial polymerization reactions, or (d) coacervation of pre-formed polymers followed by crosslinking of the thereby obtained coacervates by using a crosslinker.

Often, it is desired to transform the aqueous suspension of microcapsules into a dry powder for several reasons. First, transportation costs are reduced because of shipping less water. Second, lower energy use during transport reduces the carbon footprint. Third, an aqueous slurry requires the use of preservatives to minimize microbial activity. Fourth, an aqueous slurry requires suspension agents to maintain phase stability of the microcapsules. Fifth, the shelf life of dry powders is much longer than that of a liquid suspension of capsules. Transforming a slurry of such capsules disclosed in the art into a dry powder is challenging. Spray drying the suspension of capsules to remove water results in fracture of microcapsules due to particle-particle collisions during the drying process. Filtration followed by conductive/convective drying is a much gentler process that maintains the integrity of the microcapsules.

While others have attempted to improve the barrier properties of microcapsules, there remains significant shortcomings and limitations in the art.

For example, U.S. Pat. No. 9,944,886 B2 (“Hitchcock”) describes metal coated microcapsules with improved barrier properties. The Hitchcock metal coating is developed after the formation of the microcapsule membrane, via the use of a sterically stabilized nanosuspension of metal particle. Such metal coated microcapsules could improve barrier properties; however, it is difficult to imagine how the encapsulated active would be released, since a metal coating would be difficult to fracture. Furthermore, the processing steps involved to achieve the metal coating are laborious and expensive.

U.S. Pat. No. 4,835,224 teaches polyamines that are produced by hydrolyzing an isocyanate compound via the use of catalysts in polar organic solvents. The inventors do not contemplate the use of amino acids because these materials would degrade/denature at the reaction temperatures disclosed, and in the presence of the alkaline catalysts disclosed.

US20120157620 A1 teaches epoxy-amine polymers having primary amine, secondary amine, and hydroxyl reactive moieties. However, the inventors do not contemplate the use of amino acids to make monomers and does not contemplate the use of such monomer adducts to improve the barrier properties and dispersibility of microcapsules.

U.S. Pat. No. 4,686,242 teaches isocyanate terminated urea prepolymers that are then reacted with polyalkyl amines with equivalent weight of 500-5000 to form polyurea polyurea-polyurethane elastomers. Such polymers have high flexibility and would not be useful as reactants for microcapsule making. Moreover, the isocyanate terminated functionality makes these resins highly reactive with water. These resins are designed for immediate use in making polymeric coatings, not for making microcapsule membranes. Rather than isocyanate terminated resins, amine terminated urea resins would be better suited for use in microencapsulation.

U.S. Pat. No. 3,932,359 also teaches isocyanate terminated prepolymers by reacting lysine diisocyanate methyl ester with diols. The isocyanate terminated prepolymer is further treated with anhydrides that contain primary amines to make larger molecular weight polymers. These polymers may be useful in coatings; however, they are not useful as materials to improve barrier properties of microcapsule membranes because of their large molecular weight and poor solubility in solvents.

US 2022/0194803 A1 teaches the coating of solid particles A that may be organic or inorganic (paragraphs 16 and 17), whose surface is covered with SiO₂ units (paragraph 18), and such coated particles are then reacted with a hydrolyzed silica (paragraph 19) to yield reactive functional groups on the surface of the coated silica particles to impart water repellency benefits. The inventors do not contemplate core-shell controlled release polyurea particles comprising an active material.

U.S. Pat. No. 4,775,520 A teaches a method for preparing substantially spherical particles of a particular particle size by hydrolysis of tetra alkoxy-silane for use as a sorption material in chromatography. The inventors do not contemplate controlled release particles, rather the inventors pursue a porous particle that is able to absorb components (have high sorption).

U.S. Pat. No. 4,806,329 teaches a method of producing a granular synthetic silica comprising hydrolysis of tetra-alkoxy-silane under basic conditions and filtering said silica particles. The inventors do not contemplate low permeability particles that can be coated with silica for the purpose of controlling the release of the encapsulated active.

Accordingly, it is desired to produce low permeability particles that encapsulate benefit agents and retain such benefit agents when exposed to formulations containing solvents and surfactants. It is further desired to produce dry forms of such low permeability particles using drying processes that impart low shear to the controlled release particles. It is still further desired to provide a means to manipulate the release profile of the encapsulated active.

All references cited herein are incorporated herein by reference in their entireties. The citation of any reference is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

### BRIEF SUMMARY OF THE INVENTION

The present invention relates to microcapsules comprising a core and shell, wherein the shell comprises an interlinked multi-prepolymeric membrane developed around the core material to reduce the diffusion of core material into the environment. Materials and methods are disclosed to seal the pores in the membrane, prepare a dry form of microcapsules without fracturing shell, and redisperse dry capsules into liquid formulations.

The inventors have surprisingly found that the use of reactive amine functional urea linked amino acid isocyanate adducts and/or amine functional amino acid epoxide adducts during capsule making enhances interactions between wall forming materials to achieve a membrane with better barrier properties and better re-dispersibility of dried powder in water. The inventors have discovered that such adducts have dual functionality-they are reactive monomers that participate in the formation of the microcapsule shell, and they are surfactants that help redisperse microcapsules when incorporated into liquid formulations. The inventors have also found that the use of tetrahydroxy silanes freshly prepared from organofunctional silane in combination with amino acid adducts improves the barrier properties and improves the ease of filtration of a slurry of capsules.

Accordingly, a first aspect of the invention is a composition comprising at least one of:
(a) an amino acid isocyanate (AAI) adduct, which has a structure represented by Formula I:

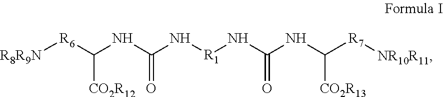

and salts thereof, and
(b) an amino acid epoxide (AAE) adduct, which has a structure represented by Formula II:

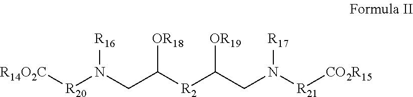

and salts thereof, where
R₁ is a moiety of a monomer or a prepolymer comprising two-NCO functional groups wherein the NCO functional groups have reacted to form urea linkages;
R₂ is a moiety of a monomer or a prepolymer comprising two glycidyl ether epoxide functional groups wherein the epoxide groups have reacted to form amino alcohol groups;
R₆, R₇, R₂₀ and R₂₁ are each independently (CH₂)ₙ, wherein n is 1-6;
R₈, R₉, R₁₀ and R₁₁ are each independently hydrogen or CH₂X;
R₁₂ and R₁₃ are each independently hydrogen, an alkali metal cation, or X;
R₁₄ and R₁₅ are each independently hydrogen, an alkali metal cation, or X;
R₁₆ and R₁₇ are each independently hydrogen or X;
R₁₈ and R₁₉ are each independently hydrogen, SiO₃ or X; and
each occurrence of X is independently selected from the group consisting of hydrogen and a substituent.

In certain embodiments, the composition comprises the AAI adduct, wherein:
R₆ and R₇ are each (CH₂)₄;
R₈, R₉, R₁₀ and R₁₁ are each hydrogen; and
R₁₂ and R₁₃ are each independently hydrogen or an alkali metal cation.

In certain embodiments, the AAI adduct is a reaction product of: (a) an amino acid or any modified form of amino acid comprising: (i) two reactive primary amines groups; (ii) two reactive secondary amines groups; or (iii) one reactive primary amine group and one reactive secondary amine group; and (b) a diisocyanate monomer or prepolymer comprising two reactive isocyanate groups, wherein the diisocyanate monomer or prepolymer is used at a stoichiometric molar ratio of isocyanate:amine of 1:2.

In certain embodiments, the amino acid comprises at least one member selected from the group consisting of lysine, arginine, ornithine, 2,4-diaminobutyric acid and lanthionine.

In certain embodiments, the diisocyanate monomer or prepolymer comprises at least one member selected from the group consisting of isophorone diisocyanate, toluene diisocyanate, hexamethylene diisocyanate, xylene diisocyanate, pentamethylene diisocyanate, 4,4′-methylene diphenyl diisocyanate and 4,4′-methylene dicyclohexyl diisocyanate.

In certain embodiments, the composition comprises the AAE adduct, wherein:
R₁₄ and R₁₅ are each independently hydrogen or an alkali metal cation;
R₁₆ and R₁₇ are each hydrogen;
R₁₈ and R₁₉ are each independently hydrogen or SiO₃; and
R₂₀ and R₂₁ are each (CH₂)₂.

In certain embodiments, the AAE adduct is a reaction product of: (a) an amino acid comprising at least one reactive amine which is chemically bonded to at least one carbon that is separated from a carbonyl carbon of a carboxylic acid of the amino acid by at least one carbon, and is one primary amine or two secondary amines; and (b) a diepoxy monomer or prepolymer having two glycidyl ether-based epoxide groups, wherein the diepoxy monomer or prepolymer is used at an equivalent ratio of epoxy:amine of 1:2.

In certain embodiments, the amino acid is at least one of beta-alanine, beta-aminobutyric acid and beta-leucine.

In certain embodiments, the diepoxy monomer or prepolymer is a member selected from the group consisting of resorcinol diglycidyl ether, bisphenol-A diglycidyl ether, bisphenol-F diglycidyl ether and hydrogenated bisphenol-A diglycidyl ether.

In certain embodiments, the composition is a polymer comprising more than one of the AAI adduct or more than one of the AAE adduct.

In certain embodiments, the composition is a copolymer comprising at least one of the AAI adduct and at least one of the AAE adduct.

A second aspect of the invention is a controlled release particle comprising: a core that comprises at least one hydrophobic active ingredient, optionally a sugar alcohol, and optionally a plasticizer; and a shell that comprises a reaction product of (a) at least one isocyanate resin and optionally an epoxy resin with (b) optionally a hydrolyzed organofunctional silane, at least one inorganic solid particle capable of coupling with a carboxylic acid group, and at least one of the AAI adduct and the AAE adduct of the invention.

In certain embodiments of the controlled release particle, the at least one hydrophobic active ingredient comprises at least one member selected from the group consisting of a flavorant, a fragrance, a chromogen, a dye, an essential oil, a sweetener, an oil, a pigment, an active pharmaceutical ingredient, a moldicide, a herbicide, a fertilizer, a phase change material, an adhesive, a vitamin oil, a vegetable oil, a triglyceride and a hydrocarbon.

In certain embodiments of the controlled release particle, the core comprises the sugar alcohol, which is an alcohol of a monosaccharide, disaccharide, trisaccharide, tetra-saccharide or penta-saccharide.

In certain embodiments of the controlled release particle, the core comprises the plasticizer, which is a member selected from the group consisting of methyl esters of rosin, polyazelate esters, di-fatty acid esters, citrate esters, polyadipate esters and polyester resins consisting of inner and intra-esters of polyhydroxy carboxylic acids.

In certain embodiments of the controlled release particle, the at least one isocyanate resin comprises a material selected from the group consisting of an aliphatic isocyanate, an aromatic isocyanate, a polymeric isocyanate, a cyclic isocyanate, a hydrophilic isocyanate, a hydrophobic isocyanate, an isocyanurate, a waterborne isocyanate and a urethane acrylate containing isocyanate functionalities.

In certain embodiments of the controlled release particle, the epoxy resin is reacted to provide the reaction product and comprises a material selected from the group consisting of an epoxidized unsaturated oil, an epoxidized vegetable oil, an epoxidized alcohol, an epoxidized silane, an epoxidized polysaccharide, a trimethylol propane triglycidyl ether, tetraglycidy ether sorbitol, multi-glycidyl ether phenol novolac, a resin containing acrylate and epoxy functional groups, a diepoxide of a cycloapliphatic alcohol, a hydrogenated Bisphenol A, and a resorcinol/bisphenol F resin with polyfunctional epoxide resin blend.

In certain embodiments of the controlled release particle, the organofunctional silane is reacted to provide the reaction product and comprises a material selected from the group consisting of hydrolyzed alkoxylated silanes, hydrolyzed trialkoxy silanes, hydrolyzed tetraalkoxylated silanes and hydrolyzed glycidyl ether silanes.

In certain embodiments of the controlled release particle, the at least one inorganic solid particle comprises a material selected from the group consisting of sodium silicate, sodium metasilicate, organically modified clay, water insoluble clay, minerals, talc, calcium carbonate, bentonite, calcium chloride, magnesium sulfate, hydroxyapatite, calcium phosphate, kaolin, montmorrilonite and amine-modified kaolin.

In certain embodiments, the controlled release particle has a diameter of 1-150 μm.

A third aspect of the invention is a consumer product comprising a plurality of the controlled release particles of the invention, wherein the consumer product is selected from the group consisting of a powdered food product, a fluid food product, a powdered nutritional supplement, a fluid nutritional supplement, a fluid fabric enhancer, a solid fabric enhancer, a fluid shampoo, a solid shampoo, a hair conditioner, a body wash, a solid antiperspirant, a fluid antiperspirant, a solid deodorant, a fluid deodorant, a fluid detergent, a solid detergent, a fluid hard surface cleaner, a solid hard surface cleaner, a fluid fabric refresher spray, a diaper, an air freshening product, a nutraceutical supplement, a controlled release fertilizer, a controlled release insecticide, a controlled release dye and a unit dose detergent further comprising a detergent and a water soluble outer film.

A fourth aspect of the invention is a method of making the inventive composition consisting of the AAI adduct, said method comprising the steps of:
(a) preparing an aqueous solution of an amino acid hydrochloride, wherein amino acids thereof comprise amines and carboxylic acids;
(b) adding an equivalent amount of an aqueous solution of sodium hydroxide to the aqueous solution of the amino acid hydrochloride to provide a modified solution;
(c) adding a diisocyanate dropwise into the modified solution kept at room temperature to 40° C.;
(d) further reacting the diisocyanate at an elevated temperature above 40° C. to complete consumption of isocyanates of the diisocyanate by the amines;
(e) adding a second equivalent amount of aqueous solution of sodium hydroxide to neutralize the carboxylic acids; and
(f) optionally filtering a product of step (e).

A fifth aspect of the invention is a method of making the inventive composition consisting of the AAE adduct, said method comprising the steps of:
(a) preparing an aqueous solution of amino acids;
(b) adding an equivalent amount of an aqueous solution of sodium hydroxide to the aqueous solution of amino acids to provide a modified solution;
(c) adding a reactive glycidyl ether based diepoxy dropwise into the modified solution kept at a temperature of 20-75° C.;
(d) further reacting epoxides of the diepoxy with amines of the amino acids at an elevated temperature of 75-85° C. to complete consumption of the epoxides by the amines; and
(e) optionally filtering the product.

A sixth aspect of the invention is a method of making the controlled release particles of the invention, said method comprising the steps of:
(a) preparing a core material by mixing the at least one hydrophobic active ingredient with the at least one isocyanate resin, optionally an epoxy resin, optionally the sugar alcohol, and optionally the plasticizer to provide a core material phase which is a solution or suspension;
(b) providing an emulsifier composition which is a homogeneous aqueous solution or aqueous dispersion of at least one emulsifier;
(c) optionally providing an amine functional low molecular weight protein or peptide;
(d) adding the core material phase into the emulsifier composition to provide an oil-in-water emulsion at room temperature to 65° C.;
(e) adding an aqueous solution of the isocyanate adduct of Formula I and/or Formula II into the oil-in-water emulsion;
(f) heating the oil-in-water emulsion from step (d) to a temperature of 35° C. to 65° C. to form a shell via an interfacial polymerization reaction;
(g) optionally adding tetrahydroxy orthosiloxane or orthosilicic acid to the oil-in-water emulsion from step (e), followed by removal of ethanol;
(h) further adding to the oil-in-water emulsion from step (f) an aqueous solution of an inorganic solid particles salt comprising at least a divalent cation capable of coupling with carboxylic acids, to provide a suspension of the controlled release particles;
(i) optionally, adding suspension or thickening agents to stabilize the microcapsules homogeneously in the slurry;
(j) optionally filtering the suspension from step (h) to form a free-flowing semi-dry powder;
(k) optionally drying further the semi-dry powder in an oven to yield a dry powder; and
(l) optionally post-curing the dry powder at an elevated temperature of 100° C. to 150° C. for 30 minutes to 60 minutes.

### BRIEF DESCRIPTION OF THE DRAWINGS

The invention will be described in conjunction with the following drawings, wherein:

FIG. 1 is a non-limiting example of a synthetic scheme for the preparation of an embodiment of an amino acid isocyanate adduct in accordance with the first aspect of the invention.

FIG. 2 is a non-limiting example of a synthetic scheme for the preparation of an embodiment of an amino acid epoxide adduct in accordance with the second aspect of the invention.

FIG. 3 is a crosslinked polymer prepared according to Examples 3A-3F.

FIG. 4 is a crosslinked polymer prepared according to Examples 4A-4B.

### DETAILED DESCRIPTION OF PREFERRED EMBODIMENTS OF THE INVENTION

### Glossary

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from the group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously.

As used herein, unless otherwise noted, the terms “capsule”, “microcapsule” and “particle” are synonyms, which refer to containers for selectively retaining an active ingredient.

As used herein, unless otherwise noted, the terms “shell,” “membrane” and “wall” are synonyms, which refer to barriers at least partially surrounding the core of the particles of the invention.

As used herein, microcapsules “formed under acidic conditions” means that part of the process of forming the microcapsule involves a step where the pH of the suspension in which the microcapsules form is adjusted into the acidic region (less than 7).

As used herein, microcapsules “formed under basic conditions” means that part of the process of forming the microcapsule involves a step where the pH of the suspension in which the microcapsules form is adjusted into the alkaline region (greater than 7).

As used herein, “an unreacted amount” refers to the amount of a reactant not used up in one or more reaction. “An unreacted amount” can be zero to any amount depending on the amount of reactants added.

As used herein, unless otherwise noted, “alkyl” whether used alone or as part of a substituent group refers to straight and branched carbon chains having 1 to 20 carbon atoms or any number within this range, for example 1 to 6 carbon atoms or 1 to 4 carbon atoms. Designated numbers of carbon atoms (e.g., C₁₋₆) shall refer independently to the number of carbon atoms in an alkyl moiety or to the alkyl portion of a larger alkyl-containing substituent. Non-limiting examples of alkyl groups include methyl, ethyl, n-propyl, isopropyl, n-butyl, sec-butyl, iso-butyl, tert-butyl, and the like. Alkyl groups can be optionally substituted. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups, the alkyl groups may be the same or different.

The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like.

The dimensions and values disclosed herein are not to be understood as being strictly limited to the exact numerical values recited. Instead, unless otherwise specified, each such dimension is intended to mean both the recited value and functionally equivalent range surrounding that value. For example, a dimension disclosed as “40 mm” is intended to mean “about 40 mm”.

As used herein “cleaning and/or treatment compositions” means products comprising fluid laundry detergents, fabric enhancers, laundry and/or rinse additives, fluid dishwashing detergents, fluid hard surface cleaning and/or treatment compositions, fluid toilet bowl cleaners that may or may not be contained in a unit dose delivery product all for consumer, agricultural, industrial or institutional use.

The term “absorbent article” is used herein in a very broad sense including any article able to receive and/or absorb and/or contain and/or retain fluids and/or exudates, especially bodily fluids/bodily exudates. Exemplary absorbent articles in the context of the present invention are disposable absorbent articles.

The term “disposable” is used herein to describe articles, which are not intended to be laundered or otherwise restored or reused as an article (i.e. they are intended to be discarded after a single use and preferably to be recycled, composted or otherwise disposed of in an environmentally compatible manner). Typical disposable absorbent articles according to the present invention are diapers, surgical and wound dressings, breast and perspiration pads, incontinence pads and pants, bed pads as well as absorbent articles for feminine hygiene like sanitary napkins, panty liners, tampons, interlabial devices or the like. Absorbent articles suitable for use in the present invention include any type of structures, from a single absorbent layer to more complex multi-layer structures. Certain absorbent articles include a fluid pervious topsheet, a backsheet, which may be fluid impervious and/or may be water vapor and/or gas pervious, and an absorbent element comprised there between, often also referred to as “absorbent core” or simply “core”.

The term “Sanitary tissue product” or “tissue product” as used herein means a wiping implement for post-urinary and/or post-bowel movement cleaning (toilet tissue products), for otorhinolaryngological discharges (facial tissue products) and/or multi-functional absorbent and cleaning uses (absorbent towels such as paper towel products and/or wipe products). The sanitary tissue products of the present invention may comprise one or more fibrous structures and/or finished fibrous structures, traditionally, but not necessarily, comprising cellulose fibers.

The term “tissue-towel paper product” refers to products comprising paper tissue or paper towel technology in general, including, but not limited to, conventional felt-pressed or conventional wet-pressed tissue paper, pattern densified tissue paper, starch substrates, and high bulk, uncompacted tissue paper. Non-limiting examples of tissue-towel paper products include towels, facial tissue, bath tissue, table napkins, and the like.

“Personal care composition” refers to compositions intended for topical application to skin or hair and can be, for example, in the form of a liquid, semi-liquid cream, lotion, gel, or solid. Examples of personal care compositions can include, but are not limited to, bar soaps, shampoos, conditioning shampoos, body washes, moisturizing body washes, shower gels, skin cleansers, cleansing milks, in-shower body moisturizers, pet shampoos, shaving preparations, etc.

“Bar soap” refers to compositions intended for topical application to a surface such as skin or hair to remove, for example, dirt, oil, and the like. The bar soaps can be rinse-off formulations, in which the product is applied topically to the skin or hair and then subsequently rinsed within minutes from the skin or hair with water. The product could also be wiped off using a substrate. Bar soaps can be in the form of a solid (e.g., non-flowing) bar soap intended for topical application to skin. The bar soap can also be in the form of a soft solid which is compliant to the body. The bar soap additionally can be wrapped in a substrate which remains on the bar during use.

“Rinse-off” means the intended product usage includes application to skin and/or hair followed by rinsing and/or wiping the product from the skin and/or hair within a few seconds to minutes of the application step.

“Ambient” refers to surrounding conditions at about one atmosphere of pressure, 50% relative humidity and about 25° C.

“Anhydrous” refers to compositions and/or components which are substantially free of added or free water.

“Antiperspirant composition” refers to antiperspirant compositions, deodorant compositions, and the like. For example, antiperspirant creams, gels, soft solid sticks, body sprays, and aerosols.

“Soft solid” refers to a composition with a static yield stress of about 200 Pa to about 1,300 Pa. The term “solid” includes granular, powder, bar and tablet product forms. The term “fluid” includes liquid, gel, paste and gas product forms.

The term “situs” includes paper products, fabrics, garments, hard surfaces, hair and skin.

The term “dry matter in the controlled release particle” refers to the mass of all ingredients in the controlled release particle after subtracting water.

The term “substantially free of” refers to 2% or less of a stated ingredient. “Free of” refers to no detectable amount of the stated ingredient or thing.

As used herein, the terms “a” and “an” mean “at least one”.

As used herein, the terms “include”, “includes” and “including” are meant to be non-limiting.

Unless otherwise noted, in discussing the commercial applications below, all component or composition levels are in reference to the active portion of that component or composition, and are exclusive of impurities, for example, residual solvents or byproducts, which may be present in commercially available sources of such components or compositions.

Similarly, all percentages and ratios are calculated by weight unless otherwise indicated and are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Adducts

The amino acid adducts of the invention are surface active monomers that are particularly useful for preparing microcapsules based on their ability to seal the pores of the microcapsule shell as well as facilitate dispersion stability of the microcapsules in the surrounding medium.

In certain embodiments, the AAI adduct comprises amine and acid functionalities and is a reaction product of an amino acid or modified amino acid comprising at least two reactive amines (two primary amines or two secondary amines or one primary amine and one secondary amine) with a diisocyanate, wherein the diisocyanate is used at a molar stoichiometric ratio of isocyanate functionality:amine functionality of 1:2. The AAI adduct has reactive primary and/or secondary amine(s), and reactive carboxylic acid functionalities. The amount of urea linked amino acid isocyanate adduct can be, e.g., from 0.25 to 30 wt. % and more specifically 2.5 wt. % or 5.0 wt. % or 7.5 wt. % or 10.0 wt. % or 10.5 wt. % or 11.5 wt. % or 13.5 wt. % or 15.0 wt. % or 17.5 wt. % or 19.5 wt. % or 21.25 wt. % or 25.0 wt. % or 30 wt. %, wherein the weight percentages are based on the weight of urea linked amino acid adduct divided by the weight of dry matter in the controlled release particle.

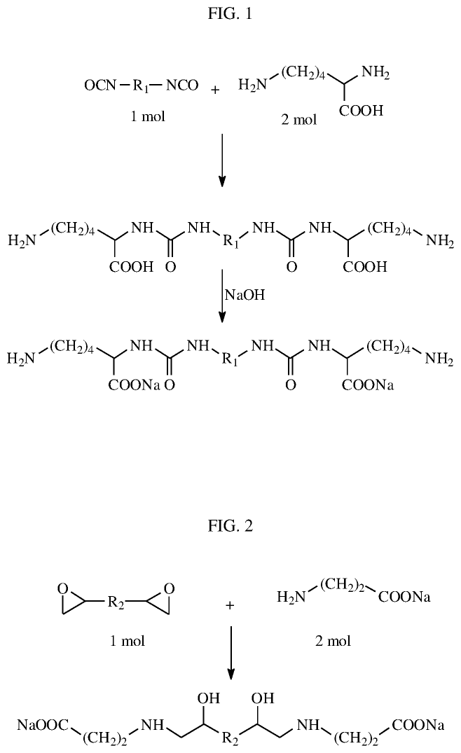

FIG. 1 shows a non-limiting example of a reaction scheme for providing an embodiment of the AAI adduct, as well as a representative chemical structure thereof.

In certain embodiments, the AAI adduct can be made using an amino acid having two reactive amine functional moieties, an isocyanate having two reactive isocyanate functional moieties, and a simple reaction chamber with the availability of overhead agitator, dropwise liquid addition option, thermocouple, heater, nitrogen protection in overhead space, distilled water, and optional 10 μm cloth filtration system.

In certain embodiments, the AAE adduct comprises secondary amine, hydroxy and acid functionalities that is a reaction product of an amino acid with at least one primary amine or two secondary amines and a diepoxide, wherein the epoxide is used at a stoichiometric ratio of epoxy functionalities to amine functionalities in an equivalent number ratio of 1:2. In epoxy-amine chemistry, epoxy equivalency and amine hydrogen equivalency are understood as: one primary NH₂ group can react with two epoxide groups while one secondary NH group can react with only one epoxide group. The AAE adduct has secondary amine hydroxyl and carboxylic acid reactive functionalities. The amount of amino acid epoxide adduct can be, e.g., from 0.25 wt. % to 20 wt. %, more specifically 2.5 wt. % or 5.0 wt. % or 7.0 wt. % or 7.5 wt. % or 7.75 wt. % or 10 wt. % or 11.50 wt. % or 12.5 wt. % or 15.0 wt. % or 17.5 wt. % or 20 wt. %, wherein the weight percentages are based on the weight of amino acid epoxy adduct divided by the weight of dry matter in the controlled release particle.

FIG. 2 shows a non-limiting example of a reaction scheme for providing an embodiment of the AAE adduct, as well as a representative chemical structure thereof.

The amino acid used to synthesize the AAI adduct preferably has two reactive amines (two primary amines, two secondary amines, or one primary and one secondary amine). The amino acid for the AAI adduct can be natural or modified. More preferably, said amino acid is selected from the group consisting of lysine, arginine, ornithine, 2,4-diaminobutyric acid and lanthionine.

The diisocyanate used to synthesize the AAI adduct is preferably selected from the group consisting of aliphatic diisocyanate, alicyclic diisocyanate, aromatic diisocyanate and mixtures thereof. More preferably, said diisocyanate is selected from the group consisting of isophorone diisocyanate, toluene diisocyanate, hexamethylene diisocyanate, xylene diisocyanate, pentamethylene diisocyanate, 4,4′-methylene diphenyl diisocyanate and 4,4′-methylene dicyclohexyl diisocyanate.

In particularly preferred embodiments, the AAI adduct is a reaction product of lysine with isophorone diisocyanate or toluene diisocyanate.

The diepoxide used to synthesize the AAE adduct is preferably at least one epoxide material having two reactive glycidyl ethers. More preferably, said diepoxide is selected from the group consisting of resorcinol diglycidyl ether, bisphenol-A diglycidyl ether, bisphenol-F diglycidyl ether and hydrogenated bisphenol-A diglycidyl ether.

The amino acid to synthesize the AAE adduct is preferably any amino acid or modified amino acid having one primary amine (NH₂) or two secondary amines (NH), wherein the NH₂ or NH is chemically bonded to carbon(s) that is/are separated from the carbonyl carbon of the carboxylic acid of the amino acid by at least one carbon (the alpha-carbon). More preferably, said amino acid is beta-alanine or beta-leucine.

In a particularly preferred embodiment, the AAE adduct is a reaction product of beta alanine with resorcinol diglycidyl ether.

In certain embodiments, the method of making the AAE adduct can comprise the use of a simple reaction chamber with the availability of overhead agitator, dropwise liquid addition option, thermocouple, heater, nitrogen protection in overhead space, distilled water, and optional 10 μm cloth Buchner funnel filtration system.

### Particles

The invention addresses one or more of the prior art deficiencies described above by providing controlled release particles. The particles are particularly well-suited for use in encapsulation of hydrophobic, nonpolar materials.

The particles are preferably used in a consumer product composition, such as, e.g., a cleaning composition, a fabric care composition and/or a personal care composition.

The controlled release particles of the present invention comprise a core and a shell.

The core comprises at least one hydrophobic active ingredient, optionally a sugar alcohol, and optionally a plasticizer.

The shell comprises a reaction product of: (a) at least one isocyanate prepolymer, (b) optionally an epoxy prepolymer, (c) at least one AAI adduct and/or at least one AAE adduct, (d) optionally a hydrolyzed organofunctional silane, and (e) an inorganic solid particle capable of coupling with a carboxylic acid group.

In certain embodiments, the organofunctional silane comprises a material selected from the group consisting of alkoxylated silanes, trialkoxy silanes, functionalized trialkoxysilanes, tetraalkoxyated silanes, 1,2 bis(triethoxysilyl) ethane, glycidyl ether silanes, and glycidoxypropyltrimethoxysilane.

In certain embodiments, the organofunctional silane as at least one member selected from the group consisting of alkoxylated silane, trialkoxy silanes, functionalized trialkoxysilanes (amino, glycidoxy, methacryloxy, vinyl), tetraalkoxylated silanes including tetramethoxy silane (tetramethoxy orthosilicate) and tetraethoxy silane (tetraethoxy orthosilicate), 1,2-bis(triethyxysilyl)ethane, glycidoxyalkyltrialkoxy silanes such as glycidoxypropyltrimethoxysilane, glycidoxypropyltriethoxysilane, glycidoxyoctyltrimethoxysilane; glycidoxyalkyl(alkyl)dialkoxy silanes such as glycidoxypropylmethyldimethoxy silane, glycidoxypropylmethyldiethoxy silane; and epoxyalkyltrialkoxy silanes such as epoxyhexyltrimethoxy silane.

The organofunctional silane is present in active encapsulated particles of the invention in an amount effective to improve the barrier properties and environmental biodegradability of the membrane. The amount of silane can be, e.g., from 0.05-15 wt. % more specifically 0.05 wt. % or 0.10 wt. % or 0.25 wt. % or 1.0 wt. % or 5.0 wt. % or 6.0 wt. % or 7.0 wt. % or 7.25 wt. % or 7.50 wt. % or 7.75 wt. % or 8.0 wt. % 9.5 wt. % or 10.0 wt. % or 12 wt. % or 14 wt. % or 15.0 wt. % wherein the weight percentages are based on the weight of dry silicate divided by the weight of active encapsulated dry matter in the controlled release particle.

In certain embodiments, the shell material of the controlled release particle may comprise inorganic solid materials. The inorganic solid particles comprise a member selected from the group consisting of sodium silicate; sodium metasilicate; organically modified or water insoluble clays, minerals, salts such as talc, calcium carbonate, bentonite, calcium chloride, magnesium sulfate, hydroxyapatite, calcium phosphate, talc, kaolin, montmorrilonite, and amine modified kaolin. Such inorganic solid particles preferably comprise a water-soluble salt. Such salt dissociates into one or more cations and one or more anions when dissolved in water. The cationic part of the salt is capable of coupling with carboxylic acids present on the surface of the core/shell particles of the invention.

Such cationic part capable of coupling with carboxylic acid of the controlled release particles of the invention contributes to the dry matter mass of the controlled release particle. Such inorganic solid particle cation is added in an amount effective to improve the barrier properties of the membrane. The amount of cationic part inorganic solid material can be, e.g., from 0.25-15 wt. % more specifically 0.25 wt. % or 0.50 wt. % or 0.75 wt. % or 1.0 wt. % or 1.25 wt. % or 1.50 wt. % or 1.75 wt. % or 1.85 wt. % or 2.0 wt. % 2.50 wt. % or 3.0 wt. % or 3.5 wt. % or 4.0 wt. % or 4.5 wt. % or 5.0 wt. % or 6.0 wt. % or 7.0 wt. % or 8.0 wt. % or 9.0 wt. % or 15 wt. %, wherein the weight percentages are based on the weight of cationic part of inorganic solid particle divided by the weight of dry matter in the controlled release particle.

In certain embodiments, the inorganic solid particle may comprise a pH change agent, such that a reaction between the inorganic particle cation or inorganic particle anion with protein in urine (for example) can result in a color change that can be an indicator of animal or human health. The presence of protein, in urine, is often an indicator of disease. Examples of inorganic solid particles that are pH change agents are copper sulfate, cupric sulfate pentahydrate, cupric sulfate hexahydrate, and the like. In certain embodiments, bicinchoninic acid or similar reagent is mixed with the dry inorganic solid particle, such that when these materials are wetted in the presence of protein, a reduction of the copper cation is enabled producing a violet color. Such color could be used as an indication for the presence of protein.

In certain embodiments, the isocyanate that reacts to form the shell material of the controlled release particles comprises a member selected from the group consisting of an aliphatic isocyanate, an aromatic isocyanate, a polymeric isocyanate, a cyclic isocyanate, a hydrophilic isocyanate, a hydrophobic isocyanate, an isocyanurate, a waterborne isocyanate and a urethane acrylate containing isocyanate functionalities.

In certain embodiments, the isocyanate comprises aliphatic isocyanates, aromatic isocyanates, polymeric isocyanates, cyclic isocyanates, hydrophilic isocyanates, hydrophobic isocyanates, waterborne isocyanates. Exemplary isocyanates are selected from the group consisting of hexamethylene diisocyanates (Desmodur H, Desmodur N3600, Desmodur N3800, Desmodur N3900, Desmodur N3200, Desmodur N3200A, Desmodur N3300, Desmodur N3400, Takenate D-170N), hexamethylene diisocyanate buret, isophorone diisocyanates (Desmodur XP2565, Desmodur Z4470), blends of hexamethylene diisocyanate and isophorone diisocyanate (Desmodur XP2847, Desmodur XP2489, Desmodur XP2838, Desmodur XP2763), pentane-1,5-diisocyanate (Stabio D-370N, Stabio D-376N), xylylene diisocyanate (Takenate 500, Takenate 600, Takenate D-110N, Takenate D-131N), polymeric methylene diphenyl diisocyanate (Mondur MR Lite), polymeric MDI (Desmodur VK 5, Desmodur VL R10, Desmodur 44V40L, Desmodur 44V70L), polyether modified hydrophilic polyisocyanates (Bayhydur XP2451/1, Bayhydur XP2547, Bayhydur XP2759, Bayhydur Ultra 304, Bayhydur Ultra 2487/1), CN9302, ionically modified isocyanates (Bayhydur 2858 XP, Bayhydur XP2759, Bayhydur eco 7190), and the like.

The isocyanate utilized in the reaction to make the shell of the particles of the invention is an amount effective to improve the barrier properties of the membrane. The amount of isocyanate can be, e.g., from 0.5-15 wt. % more specifically 0.5 wt. % or 1.0 wt. % or 1.25 wt. % or 2.5 wt. % or 5.0 wt. % to 6.0 wt. % or 7.0 wt. % or 8.0 wt. % or 10 wt. % or 12.5 wt. % or 15.0 wt. %, wherein the weight percentages are based on the weight of isocyanate divided by the weight of dry matter in the controlled release particle.

In certain embodiments, the epoxy that reacts to form the shell material of the controlled release particles comprises a member selected from the group consisting of an epoxidized unsaturated oil, an epoxidized vegetable oil, an epoxidized alcohol, an epoxidized silane, an epoxidized polysaccharide, a trimethylol propane triglycidyl ether, a resin containing acrylate and epoxy functional groups, a diepoxide of a cycloapliphatic alcohol, a hydrogenated Bisphenol A, and a resorcinol/bisphenol F resin with polyfunctional epoxide resin blend.

In certain embodiments, the epoxy is at least one member selected from the group consisting of epoxidized unsaturated oil, an epoxidized vegetable oil, an epoxidized alcohol, an epoxidized silane, an epoxidized polysaccharide, a trimethylol propane triglycidyl ether, a resin containing acrylate and epoxy functional groups, a diepoxide of a cycloapliphatic alcohol, a hydrogenated Bisphenol A, and a resorcinol/bisphenol F resin with polyfunctional epoxide resin blend, low temperature curing agents having 2 or more epoxy functional groups which are terminally located. Suitable materials include, e.g., trimethylol propane triglycidyl ether, tetraglycidyl ether sorbitol, multiglycidyl ether phenol novolac, diglycidoxy of ethylene glycol, diglycidoxy of polyethylene glycol, diglycidoxy of propylene glycol, diglycidoxy of polypropylene glycol, N,N,O-triglycidyl aminophenol, N,N,N,N-tetraglycidyl of diaminodiphenylmethane, resins containing acrylate and epoxy functional groups, diglycidoxy of the cycloapliphatic alcohol, polyglycidoxy of polyol, hydrogenated Bisphenol A, resorcinol/bisphenol F resin with polyfunctional epoxide resin blend, glycidoxypropyltrimethoxysilane, any diglycidyl and/or diglycidoxy moiety containing resins which are soluble in perfume, and any polyglycidyl or polyglycidoxy comprising resin, which are soluble in perfume.

The epoxy utilized in the reaction to make the shell of the particles of the invention is an amount effective to improve the barrier properties of the membrane. The amount of epoxy can be, e.g., from 0.05-15 wt. % more specifically 0.05 wt. % or 0.10 wt. % or 0.25 wt. % or 0.5 wt. % or 1.0 wt. % or 2.5 wt. % or 5.0 wt. % to 6.0 wt. % or 7.0 wt. % or 8.0 wt. % or 10 wt. % or 12.5 wt. % or 15.0 wt. %, wherein the weight percentages are based on the weight of isocyanate divided by the weight of dry matter in the controlled release particle.

The hydrophobic active ingredient is a hydrophobic substance that is active (or effective) to provide a desired effect, alone or in combination with other substances and/or conditions. It is present in the particles in an amount effective to provide a desired effect. The amount can be, e.g., from 30-90 wt. % or 30 wt. % or 40 wt. % to 50 wt. % or 55 wt. % or 65 wt. % or 75 wt. % or 90 wt. %, wherein the weight percentages are based on the weight of hydrophobic active divided by the weight of dry matter in the composition.

The hydrophobic active ingredient is preferably a member selected from the group consisting of a flavorant, a fragrance, a chromogen, a dye, an essential oil, a sweetener, an oil, a pigment, an active pharmaceutical ingredient, a moldicide, a herbicide, a fertilizer, a pheromone, phase change material, an adhesive, a vitamin oil, a vegetable oil, a triglyceride and a hydrocarbon.

Suitable flavorants include but are not limited to oils derived from plants and fruits such as citrus oils, fruit essences, peppermint oil, clove oil, oil of wintergreen, anise, lemon oil, apple essence, and the like. Artificial flavoring components are also contemplated. Those skilled in the art will recognize that natural and artificial flavoring agents may be combined in any sensorially acceptable blend. All such flavors and flavor blends are contemplated by this invention. Carriers may also be mixed with flavors to reduce the intensity, or better solubilize the materials. Carriers such as vegetable oils, hydrogenated oils, triethyl citrate, and the like are also contemplated by the invention.

Suitable fragrances include but are not limited to compositions comprising materials having an LogP (logarithm of octanol-water partition coefficient) of from about 2 to about 12, from about 2.5 to about 8, or even from about 2.5 to about 6 and a boiling point of less than about 280° C., from about 50° C. to about less than about 280° C., from about 50° C. to about less than about 265° C., or even from about 80° C. to about less than about 250° C.; and optionally, an ODT (odor detection threshold) of less than about 100 ppb, from about 0.00001 ppb to about less than about 100 ppb, from about 0.00001 ppb to about less than about 50 ppb or even from about 0.00001 ppb to about less than about 20 ppb. Diluents that are miscible in the fragrance oil, and act to reduce the volatility of the fragrance oil, such as isopropyl myristate, iso E super, triethyl citrate, vegetable oils, hydrogenated oils, neobee, and the like are also contemplated by the invention.

Suitable chromogens include but are not limited to Michler's hydrol, i.e. bis(p-dimethylaminophenyl) methanol, its ethers, for example the methyl ether of Michler's hydrol and the benzylether of Michler's hydrol, aromatic sulfonic and sulfinic esters of Michler's hydrol, for example the p-toluenesulfinate of Michler's hydrol, and derivatives of bis(p-dimethylaminophenyl)methylamine, e.g., N[bis(p-dimethylaminophenyl)methyl]morpholine.

Suitable dyes include but are not limited to Sudan Red 380, Sudan Blue 670, Baso Red 546, Baso Blue 688, Sudan Yellow 150, Baso Blue 645, Flexo Yellow 110, and Flexo Blue 630, all commercially available from BASF; Oil Red 235, commercially available from Passaic Color and Chemical; Morfast Yellow 101, commercially available from Morton; Nitro Fast Yellow B, commercially available from Sandoz; Macrolex Yellow 6G, commercially available from Mobay. Preferred dyes are those having good solubility in aromatic solvents.

Suitable essential oils include but are not limited to those obtained from thyme, lemongrass, citrus, anise, clove, aniseed, roses, lavender, citronella, eucalyptus, peppermint, camphor, sandalwood, cinnamon leaf and cedar. Essential oils that exhibit antimicrobial properties are also contemplated by this invention.

Suitable sweeteners include but are not limited to materials that contain varying amounts of disaccharide and/or fructose; erythritol, honey, and/or evaporated cane juice; and rebaudioside A, and the like.

Suitable pigments include but are not limited to pearl pigments of mica group such as titanium dioxide-coated mica and colored titanium dioxide-coated mica; and pearl pigments of bismuth oxychlorides such as colored bismuth oxychloride. Such pigments are available on the market under various trade names: Flamenco series (by the Mearl Corporation), TIMIRON COLORS (by MERCK) as titanium dioxide-coated mica, Timica Luster Pigments (by MEARL). Cloisonee series (by MEARL), COLORON series (by MERCK), SPECTRA-PEARL PIGMENTS (by Mallinckrodt) as colored titanium dioxide-coated mica and MIBIRON COLORS series (by MERCK) as colored bismuth oxychloride.

Suitable active pharmaceutical ingredients include but are not limited to water insoluble materials that have a melting point below 50° C.

Suitable moldicides include but are not limited to an inorganic biocide selected from the group consisting of a metal, a metal compound and combinations thereof. Preferably, the inorganic biocide is copper, cobalt, boron, cadmium, nickel, tin, silver, zinc, lead bismuth, chromium and arsenic and compounds thereof. More preferably, the copper compound is selected from the group consisting of copper hydroxide, cupric oxide, cuprous oxide, copper carbonate, basic copper carbonate, copper oxychloride, copper 8-hydroxyquinolate, copper dimethyldithiocarbamate, copper omadine and copper borate. Suitable moldicides further include but are not limited to fungicidal compounds such as, e.g., isothiazolone compounds. Typical examples of isothiazolone compounds include but not limited to: methylisothiazolinone; 5-chloro-2-methyl-4-isothiazoline-3-one, 2-methyl-4-isothiazoline-3-one, 2-n-octyl-4-isothiazoline-3-one, 4,5-dichloro-2-n-octyl-4-isothiazoline-3-one, 2-ethyl-4-isothiazoline-3-one, 4,5-dichloro-2-cyclohexyl-4-isothiazoline-3-one, 5-chloro-2-ethyl-4-isothiazoline-3-one, 2-octyl-3-isothiazolone, 5-chloro-2-t-octyl-4-isothiazoline-3-one, 1,2-benzisothiazoline-3-one, preferably 5-chloro-2-methyl-4-isothiazoline-3-one, 2-methyl-4-isothiazoline-3-one, 2-n-octyl-4-isothiazoline-3-one, 4,5-dichloro-2-n-octyl-4-isothiazoline-3-one, 1,2-benzisothiazoline-3-one, etc., more preferably 5-chloro-2-methyl-4-isothiazoline-3-one, 2-n-octyl-4-isothiazoline-3-one, 4,5-dichloro-2-n-octyl-4-isothiazoline-3-one, 1,2-benzisothiazoline-3-one, chloromethyl-isothiazolinone, 4,5-Dichloro-2-n-octyl-3 (2H)-isothiazolone and 1,2-benzisothiazolin-3-one.

Suitable herbicides include but are not limited to 2-(2-chloro-4-methylsulfonylbenzoyl)-1,3-cyclohexanedione, 2-(2-nitrobenzoyl)-4,4-dimethyl-1,3-cyclohexanedione, 2-(2-(nitrobenzoyl)-5,5-dimethyl-1,3-cyclohexanedione, and their 2-benzoylcyclohexanedione derivatives, pendimethalin, in addition to those listed in WO2006024411A2.

Suitable phase change materials include but are not limited to a crystalline alkyl hydrocarbon which is comprised of one or more crystalline straight chain alkyl hydrocarbons having 14 or more carbon atoms and heats of fusion greater than 30 cal/g. The melting and freezing point of the alkyl hydrocarbon is in the range of 0° to 80° C., preferably 5° to 50° C., and most preferably, 18° to 33° C. Representative materials are crystalline polyolefins such as polyethylene, polypropylene, polybutene, crystalline polystyrene, crystalline chlorinated polyethylene and poly(4-methylpentene-1). Crystalline ethylene copolymers such as ethylene vinylacetate, crystalline ethylene acrylate copolymers, ionomers, crystalline ethylene-butene-1 copolymers and crystalline ethylene-propylene copolymers are also useful polyolefins. Preferably, the polyolefins are crosslinked such that they are form stable upon heating above their crystalline melting point.

Suitable adhesives include but are not limited to compositions comprising an elastomer and a tackifying agent. The elastomer adds toughness to the adhesive film and also is responsible for at least part of the required initial pressure-sensitive tackiness. The elastomeric materials are water insoluble and are inherently tacky or are capable of being rendered tacky by mixture with compatible tackifying resins. Preferably, the elastomers are natural rubber or butadiene or isoprene synthetic polymers or copolymers such as butadiene-isobutylene copolymers, butadiene-acrylonitrile copolymers, butadiene-styrene copolymers, polychloroprene or similar elastomers. A combination of the above elastomers may be utilized. Preferred tackifying agents include unsaturated natural resins such as rosin or derivatives thereof, such as rosin esters of polyols such as glycerol or pentaerythritol, hydrogenated rosins or dehydrogenated rosins.

Suitable vitamin oils include but are not limited to fat-soluble vitamin-active materials, provitamins and pure or substantially pure vitamins, both natural and synthetic, or chemical derivatives thereof, crude extractions containing such substances, vitamin A, vitamin D, and vitamin E active materials as well as vitamin K, carotene and the like, or mixtures of such materials. The oil-soluble vitamin oil concentrate may be a high potency fish liver oil containing vitamin A and/or D, a synthetic vitamin A palmitate and/or acetate concentrated in an oil solution, vitamin D, or D either concentrated in oil solution or as an oleaginous resin, vitamin E (d-alpha tocopheryl acetate) in an oil solution, or vitamin K in oil solution, or beta-carotene as a crystalline oil suspension in oil.

Suitable vegetable oils include but are not limited to oils derived from palm, corn, canola, sunflower, safflower, rapeseed, castor, olive, soybean, coconut and the like in both the unsaturated forms and hydrogenated forms, and mixtures thereof.

Suitable triglycerides include but are not limited to those disclosed in U.S. Pat. No. 6,248,909B1.

Suitable hydrocarbons that can be the active or can be used in combination with the active in order to change the physical or chemical properties of the active, include but are not limited to, waxes, density modifiers, surface tension modifiers, melting point modifiers, viscosity modifiers, and mixtures thereof. Examples include animal waxes such as beeswax, plant waxes such as carnauba wax, candelilla wax, bayberry wax, castor wax, tallow tree wax, soya wax, rice bran wax, hydrogenated rice bran wax, soya wax, hydrogenated soya wax, hydrogenated vegetable oil. Examples of petroleum derived waxes are paraffin waxes and microcrystalline waxes. An example of synthetic wax is polyethylene wax. Examples of materials that can modify the density of the active phase in the particle are brominated vegetable oil, nanoclays such as montmorrilonite or kaolin, hydrophobically modified clays, hydrophobically modified precipitated silicas or fumed silicas. Examples of oil thickening agents are waxes mentioned above, modified organopolysiloxanes, silicone gums, hydrogenated castor oil, paraffin oils, polyolefins, and the like.

In certain embodiments, a plasticizer is included in the oil phase and is at least one member selected from the group consisting of methyl esters of rosin, polyazelate esters, di-fatty acid esters, citrate esters, polyadipate esters and polyester resins consisting of inner and intra-esters of polyhydroxy carboxylic acids.

The amount of plasticizer can be, e.g., from 0.025-2.0 wt. % more specifically 0.025 wt. % or 0.05 wt. % or 0.10 wt. % or 0.15 wt. % or 0.20 wt. % or 0.25 wt. % or 0.50 wt. % or 0.75 wt. % or 0.85 wt. % or 1.0 wt. % or 1.25 wt. % or 1.50 wt. % or 1.75 wt. % or 2.0 wt. %, wherein the weight percentages are based on the weight of plasticizer divided by the weight of dry matter in the controlled release particle.

In certain embodiments, a sugar alcohol is included in the oil phase and is at least one member selected from the group consisting of monosaccharide, disaccharide, trisaccharide, tetra-saccharide or penta-saccharide.

The sugar alcohol is present in particles of the invention in an amount effective to improve the barrier properties of the membrane. The amount of sugar alcohol can be, e.g., from 0.025-2.0 wt. % more specifically 0.025 wt. % or 0.10 wt. % or 0.15 wt. % or 0.20 wt. % or 0.25 wt. % or 0.50 wt. % or 0.75 wt. % or 0.85 wt. % or 1.0 wt. % or 1.25 wt. % or 1.50 wt. % or 1.75 wt. % or 2.0 wt. %, wherein the weight percentages are based on the weight of sugar alcohol divided by the weight of dry matter in the controlled release particle.

The controlled release particles are preferably spherical but non-spherical shapes are also within the scope of the invention. The particles preferably have a diameter from 0.05-250 microns, or from 0.1 microns to less than 100 microns.

### Method of Making the Particles

The controlled release particles of the invention preferably comprise a core and a polymeric shell, wherein the core comprises: (1) at least one hydrophobic active ingredient and (2) the polymeric shell (interlinked membrane) is obtained by an interfacial polymerization reaction among reactive isocyanate forming a polyurea moiety or segment, glycidyl ether based epoxy forming crosslinked epoxy-amine moiety or segment, amine and acid functional urea linked amino acid isocyanate adduct, amine and hydroxy and acid functional amino acid epoxide adduct, optionally a hydroxy silane forming crosslinked silicate moiety or segment, and inorganic solid particle having at least a cation capable of forming coacervate layer by coupling with carboxylic acid groups. Preferably, the amine co-reactant with isocyanate or epoxy is a low molecular weight protein or peptide that is water soluble or dispersible.

Not to be limited by theory, polyurea shell of capsules are made via interfacial polymerization at the oil-water interface via a chemical reaction between prepolymers and/or monomers dissolved or dispersed in the oil phase with monomers and/or prepolymers dissolved or dispersed in the aqueous phase. The choice of monomers, crosslinking conditions, surfactants, emulsifiers, and other additives used influences the permeability properties of the membrane.

Not to be limited by theory, the amine-acid functional amino acid isocyanate adducts and amine-hydroxy-acid functional amino acid epoxide adducts added in the aqueous phase can react with several functional groups present in the hydrophobic active phase, including reactive isocyanates, and reactive epoxies. The reactivity of amine is higher with isocyanate, and therefore provides a membrane via an interfacial polymerization reaction that limits the diffusion of the hydrophobic oil phase into the surrounding aqueous phase. Not to be limited by theory, the amine moieties in the amino acid adducts can also react with epoxy functionality to form amino alcohols. The amino alcohols can in turn react with the isocyanates, such reaction will lead to the possible formation of polyurethanes and polyureas which will reduce the permeability of the membrane. Introduction of the tetrahydroxy orthosilanes can further crosslink and or interlink preformed polyurea-polyurethane-epoxy-amine membrane or form a cage like structure in the membrane comprising Si—O—Si bonds to reduce the permeability of the membrane. In addition, acid functionality (in amino acid adducts) on preformed capsule shell (formed from the reaction among isocyanate, epoxy, amino acid adducts, hydroxy silane) surface reacts with cationic part of inorganic salt to form a coacervate layer which further reduces the permeability of the membrane.

Controlled release particles of the present invention are made using the following protocol: 1) preparing a core material phase by mixing the at least one hydrophobic active material with the at least one isocyanate resin, the optional epoxy resin, the optional sugar alcohol, and the optional plasticizer to make a solution or suspension; 2) making a homogeneous aqueous solution or aqueous dispersion of the at least one emulsifier and optionally the at least one low molecular weight protein; 3) adding the core material phase into the aqueous solution or dispersion to prepare an oil-in-water (O/W) emulsion at room temperature to 65° C.; 4) adding aqueous solution of amine-acid functional urea linked amino acid isocyanate adduct, and amine-hydroxy-acid functional amino acid epoxide adduct into prepared emulsion; 5) heating the emulsion to a temperature of from 35° C. to 65° C. to form a shell via an interfacial polymerization reaction; 6) optionally adding the freshly prepared tetrahydroxy orthosilicic acid from the hydrolysis of tetraethoxy orthosiloxane; 7) further adding aqueous solution of an inorganic salt having at least a divalent cation capable of coupling with carboxylic acid; 8) optionally filtering the suspension of microcapsules to form free flowing semi dry powder; 9) optionally, drying further the semi dry powder in an oven to yield a dry powder; 10) optionally, post curing the dry powder at elevated temperatures (100° C. to 150° C.) for 30 minutes to 60 minutes.

The emulsifier is present in the suspension, on a dry basis (weight of emulsifier per weight of dry matter in the suspension), of the invention in an amount effective to achieve the desired particle size distribution. The amount can be, e.g., from 0.025-5 wt. % more specifically 0.025 wt. % or 0.05 wt. % 0.075 wt. % or 0.10 wt. % or 0.15 wt. % or 0.20 wt. % or 0.30 wt. % or 0.40 wt. % or 0.50 wt. % or 0.75 wt. % or 1.0 wt. % or 1.5 wt. % or 2.0 wt. % or 2.5 wt. % or 3.0 wt. % 3.5 wt. % or 4.0 wt. % or 4.5 wt. % or 5.0 wt. % or not greater than 10 wt. %.

Emulsifiers or surfactants of all types are suitable for use in the practice of the present process though it is to be appreciated, and those skilled in the art will readily recognize that different systems, e.g., different core monomer and/or core materials, will be better suited with one or more classes of emulsifiers than others. Specifically, while the present teachings are applicable to anionic, cationic, non-ionic and amphoteric emulsifiers generally, preferred emulsifiers are non-ionic emulsifiers, particularly those having polyalkylether units, alkylphenylether units especially polyethylene oxide units, with degrees of polymerization of the alkylene ether unit of greater than about 6. Preferred emulsifiers are those which significantly reduce the interfacial tension between the continuous aqueous phase and dispersed oil phase composition, and thereby reduce the tendency for droplet coalescence. In this regard, generally the emulsifiers for use in the aqueous phase for aiding in the oil in water emulsion or dispersion will have HLB values of from 10 to 18. Of course, emulsifiers/surfactants of lower and higher HLB values that achieve the same objective as noted are also included.

Exemplary emulsifiers include, but are not limited to gums such as acacia gum, gum arabic, konjac gum, and xantham gum; poly(meth)acrylic acids and derivatives. Most preferably, the emulsifier/emulsion stabilizer is a polyvinyl pyrrolidone, copolymers of polyvinyl pyrrolidone with vinyl acetate, vinyl alcohol, vinyl imidazole; polyglycerol oleates; polyvinyl alcohol; ethoxylate nonylphenol; water soluble proteins such as gelatin, whey protein, pea protein, and the like; modified polysaccharides such as octenyl succinate starch; Pickering emulsion stabilizers such as colloidal silica; and chitosan.

Additional exemplary anionic surfactants and classes of anionic surfactants suitable for use in the practice of the present invention include: sulfonates; sulfates; sulfosuccinates; sarcosinates; alcohol sulfates; alcohol ether sulfates; alkylaryl ether sulfates; alkylaryl sulfonates such as alkylbenzene sulfonates and alkylnaphthalene sulfonates and salts thereof; alkyl sulfonates; mono- or di-phosphate esters of polyalkoxylated alkyl alcohols or alkylphenols; mono- or di-sulfosuccinate esters of C12 to C15 alkanols or polyalkoxylated C12 to C15 alkanols; ether carboxylates, especially alcohol ether carboxylates; phenolic ether carboxylates; polybasic acid esters of ethoxylated polyoxyalkylene glycols consisting of oxybutylene or the residue of tetrahydrofuran; sutfoalkylamides and salts thereof such as N-methyl-N-oleoyltaurate Na salt; polyoxyalkylene alkylphenol carboxylates; polyoxyalkylene alcohol carboxylates alkyl polyglycosidelalkenyl succinic anhydride condensation products; alkyl ester sulfates; naphthalene sulfonates; naphthalene formaldehyde condensates; alkyl sulfonamides; sulfonated aliphatic polyesters; sulfate esters of styrylphenyl alkoxylates; and sulfonate esters of styrylphenyl alkoxylates and their corresponding sodium, potassium, calcium, magnesium, zinc, ammonium, alkylammonium, diethanolammonium, or triethanolammonium salts; salts of ligninsulfonic acid such as the sodium, potassium, magnesium, calcium or ammonium salt; polyarylphenol polyalkoxyether sulfates and polyarylphenol polyalkoxyether phosphates; and sulfated alkyl phenol ethoxylates and phosphated alkyl phenol ethoxylates; sodium lauryl sulfate; sodium laureth sulfate; ammonium lauryl sulfate; ammonium laureth sulfate; sodium methyl cocoyl taurate; sodium lauroyl sarcosinate; sodium cocoyl sarcosinate; potassium coco hydrolyzed collagen; TEA (triethanolamine) lauryl sulfate; TEA (Triethanolamine) laureth sulfate; lauryl or cocoyl sarcosine; disodium oleamide sulfosuccinate; disodium laureth sulfosuccinate; disodium dioctyl sulfosuccinate; N-methyl-N-oleoyltaurate Na salt; tristyrylphenol sulphate; ethoxylated lignin sulfonate; ethoxylated nonylphenol phosphate ester calcium alkylbenzene sulfonate; ethoxylated tridecylalcohol phosphate ester, dialkyl sulfosuccinates; perfluoro (C6-C18)alkyl phosphonic acids; perfluoro (C6-C18)alkyl-phosphinic acids; perfluoro (C3-C20)alkyl esters of carboxylic acids; alkenyl succinic acid diglucamides; alkenyl succinic acid alkoxylates; sodium dialkyl sulfosuccinates; and alkenyl succinic acid alkylpolyglykosides. Further exemplification of suitable anionic emulsifiers include, but are not limited to, water-soluble salts of alkyl sulfates, alkyl ether sulfates, alkyl isothionates, alkyl carboxylates, alkyl sulfosuccinates, alkyl succinamates, alkyl sulfate salts such as sodium dodecyl sulfate, alkyl sarcosinates, alkyl derivatives of protein hydrolyzates, acyl aspartates, alkyl or alkyl ether or alkylaryl ether phosphate esters, sodium dodecyl sulphate, phospholipids or lecithin, or soaps, sodium, potassium or ammonium stearate, oleate or palmitate, alkylarylsulfonic acid salts such as sodium dodecylbenzenesuifonate, sodium dialkylsulfosuccinates, dioctyl sulfosuccinate, sodium dilaurylsulfosuccinate, poly(styrene sulfonate) sodium salt, alkylene-maleic anhydride copolymers such as isobutylene-maleic anhydride copolymer, or ethylene maleic anhydride copolymer gum arabic, sodium alginate, carboxymethylcellulose, cellulose sulfate and pectin, poly(styrene sulfonate), pectic acid, tragacanth gum, almond gum and agar; semi-synthetic polymers such as carboxymethyl cellulose, sulfated cellulose, sulfated methylcellulose, carboxymethyl starch, phosphated starch, lignin sulfonic acid; maleic anhydride copolymers (including hydrolyzates thereof), polyacrylic acid, polymethacrylic acid, acrylic acid alkyl acrylate copolymers such as acrylic acid butyl acrylate copolymer or crotonic acid homopolymers and copolymers, vinylbenzenesulfonic acid or 2-acrylamido-2-methylpropanesulfonic acid homopolymers and copolymers, and partial amide or partial ester of such polymers and copolymers, carboxymodified polyvinyl alcohol, sulfonic acid-modified polyvinyl alcohol and phosphoric acid-modified polyvinyl alcohol, phosphated or sulfated tristyrylphenol ethoxylates.

Exemplary amphoteric and cationic emulsifiers include alkylpolyglycosides; betaines; sulfobetaines; glycinates; alkanol amides of C8 to C18 fatty acids and C8 to C18 fatty amine polyalkoxylates; C1 to C18 alkyldimethylbenzylammonium chlorides; coconut alkyldimethylaminoacetic acids: phosphate esters of C8 to C18 fatty amine polyalkoxylates; alkylpolyglycosides (APG) obtainable from an acid-catalyzed Fischer reaction of starch or glucose syrups with fatty alcohols, in particular C8 to C18 alcohols, especially the C8 to C10 and C12 to C14 alkylpolyglycosides having a degree of polymerization of 1.3 to 1.6, in particular 1.4 or 1.5. Additional cationic emulsifiers include quaternary ammonium compounds with a long-chain aliphatic radical, e.g. distearyldiammonium chloride, and fatty amines. Among the cationic emulsifiers which may be mentioned are alkyldimethylbenzylammonium halides, alkyldimethylethyl ammonium halides, etc. specific cationic emulsifiers include palmitamidopropyl trimonium chloride, distearyl dimonium chloride, cetyltrimethylammonium chloride, 1H-Imidazolium, 1-ethenyl-3-methyl-, chloride, polymer with 1-ethenyl-2-pyrrolidinone, and polyethyleneimine. Additional amphoteric emulsifiers include alkylaminoalkane carboxylic acids betaines, sulphobetaines, imidazoline derivatives, lauroamphoglycinate, sodium cocoaminopropionate, and the zwitterionic emulsifier cocoamidopropyl betaine.

Suitable non-ionic emulsifiers are characterized as having at least one non-ionic hydrophilic functional group. Preferred non-ionic hydrophilic functional groups are alcohols and amides and combinations thereof. Examples of non-ionic emulsifiers include: mono and diglycerides; polyarylphenol polyethoxy ethers; polyalkylphenol polyethoxy ethers; polyglycol ether derivatives of saturated fatty acids; polyglycol ether derivatives of unsaturated fatty acids; polyglycol ether derivatives of aliphatic alcohols; polyglycol ether derivatives of cycloaliphatic alcohols; fatty acid esters of polyoxyethylene sorbitan; alkoxylated vegetable oils; alkoxylated acetylenic diols; polyalkoxylated alkylphenols; fatty acid alkoxylates; sorbitan alkoxylates; sorbitol esters; C8 to C22 alkyl or alkenyl polyglycosides; polyalkoxy styrylaryl ethers; amine oxides especially alkylamine oxides; block copolymer ethers; polyalkoxylated fatty glyceride; polyalkylene glycol ethers; linear aliphatic or aromatic polyesters; organo silicones; sorbitol ester alkoxylates; ethoxylated castor oil; amides of fatty acids such as stearamide, lauramide diethanolamide, and lauramide monoethanolamide; aryl ethers of polyoxyalkylene glycols such as polyoxyethylene glycol nonylphenyl ether and polypropylene glycol stearyl ether. Also preferred as non-ionic emulsifiers are various latex materials, stearates, and lecithins.

Further exemplary emulsifiers that are available in anionic, nonionic, and cationic forms are colloidal silica suspensions that comprise a suspension of silica particles having a particle size from about 8 nanometers to 40 nanometers. Laponite and bentonite clays can also be exfoliated to nanometer size using high shear to make suspensions in water. Such inorganic solid particle suspensions can be used as emulsifiers to make Pickering oil-in-water emulsions.

In a preferred embodiment of the invention, the aqueous suspension of controlled release particles is dried into a powder. The addition of preferred amount of tetra hydroxy orthosiloxane followed by certain amount aqueous solution of inorganic salt (e.g. calcium chloride) having at least a divalent cation results in capsule surface hard and rigid that facilitates filtration of the controlled release particles. Filtration can be done using a Buchner funnel and vacuum pump. On a commercial scale, filtration can be achieved using Oberlin pressure filtration apparatus. The filtered semi dried free flowing powder can then be further dried using an oven with trays of powder, or at commercial scale a conveyor dryer with infrared heating, a conveyor dryer with convective heating, or fluid bed drying.

In another embodiment of the method of making controlled release particles, the particles comprise amine, hydroxy and acid functional amino acid epoxide adduct (AAE), amine and acid functional urea linked amino acid isocyanate adduct (AAI), and low molecular weight protein (e.g. whey protein) of highly water dispersibility or water solubility. Said method of making may comprise the following steps: i) preparing a core material phase by mixing the at least one hydrophobic active material with the at least one isocyanate prepolymer or resin, the optional one epoxy prepolymer or resin, the optional sugar alcohol, the optional plasticizer to make a suspension or solution; ii) adding the core material phase into an aqueous phase (maintained at room temperature to 35° C. to 40° C. to 60° C. to 65° C. (temperature selection depends on the hydrophobic active materials) comprising protein, surfactant and or emulsifier, and an optional inorganic salt to prepare an oil-in-water (O/W) emulsion under agitation; iii) addition of aqueous solution of amine and acid functional urea linked amino acid isocyanate adduct (AAI), and or amine, hydroxy and acid functional amino acid epoxide adduct (AAE); iv) heating the emulsion to a temperature from 35° C. to 40° C. to 60° C. to 65° C. to form a shell via an interfacial polymerization reaction; and v) addition of suspension stabilizers (xanthan gum, gum arabic, magnesium sulfate, naphthalene sulfonate etc.) as per requirements.

### Compositions Containing the Particles

The invention further comprises compositions (e.g., products, articles of manufacture, etc.) comprising the controlled release particles. Such compositions include but are not limited baby care, beauty care, fabric & home care, family care, feminine care, health care, snack and/or beverage products or devices intended to be used or consumed in the form as sold, and not intended for subsequent commercial manufacture or modification. Such products include but are not limited to fine fragrances (e.g., perfumes, colognes eau de toilettes, after-shave lotions, pre-shave, face waters, tonics, and other fragrance-containing compositions for application directly to the skin), diapers, bibs, wipes; products for and/or methods relating to treating hair (human, dog, and/or cat), including, bleaching, coloring, dyeing, conditioning, shampooing, styling; deodorants and antiperspirants; personal cleansing; cosmetics; skin care including application of creams, lotions, and other topically applied products for consumer use; and shaving products, products for and/or methods relating to treating fabrics, hard surfaces and any other surfaces in the area of fabric and home care, including: air care, car care, dishwashing, fabric conditioning (including softening), laundry detergency, laundry and rinse additive and/or care, hard surface cleaning and/or treatment, toilet care and cleaning, and other cleaning for consumer or institutional use; products and/or methods relating to bath tissue, facial tissue, paper handkerchiefs, and/or paper towels; tampons, feminine napkins; products and/or methods relating to oral care including toothpastes, tooth gels, tooth rinses, denture adhesives, tooth whitening; over-the-counter health care including cough and cold remedies, pain relievers, prescription pharmaceuticals, pet health and nutrition, and water purification; processed food products intended primarily for consumption between customary meals or as a meal accompaniment (non-limiting examples include potato chips, tortilla chips, popcorn, pretzels, corn chips, cereal bars, vegetable chips or crisps, snack mixes, party mixes, multigrain chips, snack crackers, cheese snacks, pork rinds, com snacks, pellet snacks, extruded snacks and bagel chips); and coffee. Moreover, such products include, but are not limited to, a powdered food product, a fluid food product, a powdered nutritional supplement, a fluid nutritional supplement, a fluid fabric enhancer, a solid fabric enhancer, a fluid shampoo, a solid shampoo, hair conditioner, body wash, solid antiperspirant, fluid antiperspirant, solid deodorant, fluid deodorant, fluid detergent, solid detergent, fluid hard surface cleaner, solid hard surface cleaner, a fluid fabric refresher spray, a diaper, an air freshening product, a nutraceutical supplement, a controlled release fertilizer, a controlled release insecticide, a controlled release dye, and a unit dose detergent comprising a detergent and the controlled release particles in a water soluble film.

Fluid compositions of the invention preferably further comprise at least one suspension agent to suspend the controlled release particles, wherein the at least one suspension agent is at least one member selected from the group consisting of a rheology modifier, a structurant and a thickener. The at least one suspension agent preferably has a high shear viscosity at, 20 sec⁻¹ shear rate and at 21° C., of from 1 to 7000 cps and a low shear viscosity, at 0.5 sec⁻¹ shear rate and at 21° C., of greater than 1000 cps or 1000-200,000 cps. In certain embodiments, the composition has a high shear viscosity, at 20 sec⁻¹ and at 21° C., of from 50 to 3000 cps and a low shear viscosity, at 0.5 sec⁻¹ shear rate and at 21° C., of greater than 1000 cps or 1000-200,000 cps.

Preferably, the at least one suspension agent is selected from the group consisting of polyacrylates, polymethacrylates, polycarboxylates, pectin, alginate, gum arabic, carrageenan, gellan gum, xanthan gum, guar gum, gellan gum, glycerol, dipropylene glycol, polypropylene glycol, hydroxyl-containing fatty acids, hydroxyl-containing fatty esters, hydroxyl-containing fatty waxes, castor oil, castor oil derivatives, hydrogenated castor oil derivatives, hydrogenated castor wax and mixtures thereof.

The invention will be illustrated in more detail with reference to the following Examples, but it should be understood that the present invention is not deemed to be limited thereto.

### EXAMPLES

### Materials and Methods

The following is a representative perfume oil composition used for capsule making.

TABLE 1
Perfume oil composition

| Material | wt. % | Functionality |
|---|---|---|
| CITRONELLYL NITRILE | 1.00% | NITRILE |
| TRIPLAL | 0.25% | ALDEHYDE |
| FLORHYDRAL | 0.10% | ALDEHYDE |
| ALDEHYDE C-10 | 0.10% | ALDEHYDE |
| ALDEHYDE C-12 LAURIC | 0.20% | ALDEHYDE |
| ALLYL CYCLOHEXYL PROPIONATE | 1.00% | ESTER |
| CETALOX | 0.20% | FURAN |
| ANISIC ALDEHYDE | 0.10% | ALDEHYDE |
| CYCLACET | 10.00% | ESTER |
| CYCLAPROP | 5.00% | ESTER |
| DIHYDROMYRCENOL | 10.00% | ALCOHOL |
| DIPHENYL OXIDE | 1.00% | OXIDE |
| HABANOLIDE | 2.50% | KETONE |
| YARA YARA | 2.00% | ETHER |
| CIS-3-HEXENYL SALICYLATE | 2.00% | ESTER |
| VERDOX | 2.50% | ESTER |
| HEXYLCINNAMIC ALDEHYDE | 20.00% | ALDEHYDE |
| BHT | 0.50% | 0.0025 |
| ISO E SUPER | 2.50% | KETONE |
| KOAVONE | 2.50% | 0.0625 |
| EUCALYPTOL | 0.20% | ALCOHOL |
| MANZANATE, 10% IPM | 0.50% | ESTER |
| MUSCENONE, 10% IPM | 0.50% | KETONE |
| LAEVO CARVONE, 10% IPM | 0.50% | 0.0025 |
| METHYL ANTHRANILATE | 0.10% | ESTER |
| METHYL IONONE GAMMA | 1.25% | KETONE |
| LILIAL | 10.00% | ALDEHYDE |
| ALDEHYDE C-12 MNA, 10% DPG | 0.50% | ALDEHYDE |
| MYRAC ALDEHYDE | 0.50% | ALDEHYDE |
| D-LIMONENE | 5.00% | TERPENE |
| PEONILE | 2.50% | NITRILE |
| ETHYLENE BRASSYLATE | 12.50% | ESTER |
| PHENOXANOL | 2.50% | ALCOHOL |

### Scanning Electron Microscopy

A Phenom Pure (Nanoscience Instruments Model PW-100-019) Scanning Electron Microscope is used to understand the particle morphology, and nature of particle deposits on fabrics. PELCO tabs carbon tape (12 mm OD, Ted Pella product number 16084-1) is applied to an aluminum specimen mount (Ted Pella Product No 16111). Next, the powder sample is placed onto the carbon tape using a transfer spatula. Excess powder is removed by blowing Dust-Off compressed gas onto the sample. The stub is then left in a desiccator under vacuum for 16 hours to flash off any volatiles. The sample is then placed into the Phenom Pure, and imaged to visualize particle morphology.

### Leakage Test

### Standards

Prepare a 50.0 mg+0.5 stock solution of Dimethyl Phthalate standard in 20.0 mL of hexane. Prepare the internal standard in 20.0 mL glass crimp vial.

Prepare a five-point calibration curve and control standard in Hexanes. The control standard is used to verify accuracy of the calibration curve. See Table 2 for standard preparation.

TABLE 2
Table 2 Standard Calibration Curve in Hexanes

| | | | | |
|---|---|---|---|---|
| | Perfume | Target | Record Actual | Hexane |
| Standard ID | Name | Mass (mg) | Weight (mg) | (mL) |

| | | |
|---|---|---|
| Sample ID-FOH01 | 5 to 8 mg | 10.0 |
| Sample ID-FOH02 | 10 to 13 mg | 10.0 |
| Sample ID-FOH03 | 16 to 19 mg | 10.0 |
| Sample ID-FOH04 | 24 to 28 mg | 10.0 |
| Sample ID-FOH05 | 45 to 52 mg | 10.0 |
| Sample ID-FOH06 | 18 to 21 mg | 10.0 |

### Sample Analysis

Weigh out a known amount of solid perfume sample on analytical balance into a 40 mL glass bottle. Record the weight in grams. The amount of material (grams) is calculated by formulated percentage of perfume oil inside the capsules. The final weight of sample (recorded in mg) will be used to calculate the % Free Perfume Oil.

Using a top loader balance, add 10 grams of 1% uric acid solution to sample and standards.

Place the samples and standards on platform shaker, secure the vials, and set the speed to 225-235 RPM for 30 minutes.

Pipette 10.0 mL of Hexanes to each sample and standards. Gently invert the sample 10 times.

Using a top loader balance, add 2-4 grams of sodium chloride to samples and standards to achieve better phase separation.

Place the standards and samples on platform shaker, secure the vials, and set the speed to 225-235 RPM for 10 minutes.

After mixing, centrifuge the samples and standards at speed of 2800 RPM for 2 minutes.

Carefully remove the samples from centrifuge without disturbing the layers. Observe if there is a good separation. Additional sodium chloride or longer time in centrifuge may be required if poor separation is observed.

Using a disposable pipette to transfer supernatant liquid to a 5 mL disposable syringe with 0.45 μM syringe filter. Only transfer 4-5 mL of hexane layer. Carefully filter the sample. Do not force the plunger too hard or there might be possible liquid or solids that might be in filtrate. Avoid transferring any solid material or water into the vial.

Pipette 980 μL (Sample, Standards, and Solvent Blanks) and 20 μL of internal standard into 2 mL autosampler.

If necessary, dilution of samples can be made in order to bring the sample concentration (mg) within the range of linear curve. Any samples outside the standard concentration (mg) must be repeated.

TABLE 3
GC CONDITIONS
Gas Chromatography/Mass Spectrophotometer Conditions

| Capillary Column | DB-5MS, 30 meter, 0.25 μm film, | |
|---|---|---|
| | ID = 0.25 mm | |
| Carrier Gas | UHP Helium, 1.2 mL/min through | |
| | the column | |
| Injection Volume | 1.0 μL, Split, Split Ratio 8.0:1 | |
| Injector Port Temperature | 250° | C. |

Oven Conditions

| | | |
|---|---|---|
| Initial Temperature | 40° | C. |
| Hold Time | 2 | minutes |
| Ramp | 5° | C./min |
| Final Temperature | 270° | C. |
| Final Hold | 6 | minutes |
| Total Run Time | 54.0 | minutes |

Mass Spectrophotometer Detector Conditions

| MS Source Temperature | 230° | C. |
|---|---|---|
| MS Quad | 150° | C. |
| Back Detector | 270° | C. |
| Tune File | Atune.u | |
| Scan Range | 40 to 600 | amu |
| Solvent Delay | 4.5 | minutes |

Calculate the average Response Factor of total area sum from standard calibration curve. See equation below:

$$RF = ( ( Ax ) * ( Cis ) / ( ( Ais ) ( Cx ) )$$

where:
AX=Area of the compound
CX=Standard Concentration (mg)
Ais=Area of the internal standard
Cis=Internal Standard Concentration (mg)

Calculate sample concentration (mg). See equation below:

$$CXs = ( ( ( Ax ) * ( Cis ) / ( ( Ais ) ( RFAVE ) ) ) * df$$

where:
AX=Area of the compound
CXs=Sample Concentration (mg)
Ais=Area of the internal standard
RFAVE=Average Response Factor
df=Sample Dilution

By using the sample concentration (mg) of perfume oil found in the hexane extract and dividing by the theoretical perfume dosed into the uric acid solution, one can calculate the amount of perfume that has leached out of the microcapsule. This is reported as % Leakage.

### Example 1—Amine and Acid Functional Urea Linked Amino Acid Isocyanate Adducts

### Example 1A

Weigh out 285 g water into a reaction kettle. Add 163 g lysine hydrochloride under agitation at room temperature. To this solution, add 89.24 g 40% sodium hydroxide aqueous solution under agitation. Continue agitation until temperature come down to nearly 25° C. Add 99.06 g isophorone diisocyanate under agitation at room temperature. Raise temperature to 35-40° C. and continue reaction for 2 hours. Set temperature to 60° C. and continue reaction for 2 hours. Cool down to 30° C. under agitation and add 89.24 g 40% sodium hydroxide solution. The product, amine terminated diamino-diurea-disodium carboxylate of 34.40% in water was synthesized by this technique and abbreviated here as X1.

### Example 1B

Weigh out 260 g water into a reaction kettle. Add 50 g lysine hydrochloride under agitation at room temperature. To this solution, add 27.37 g 40% sodium hydroxide aqueous solution under agitation. Continue agitation until temperature comes down to nearly 25° C. Add 23.84 g toluene diisocyanate dropwise under agitation. Addition of toluene diisocyanate raised temperature to 35-40° C. Continue reaction for 2 hours at 40° C. Set temperature to 60° C. and continue reaction for 2 hours. Cool down to 50° C. under agitation and add 27.37 g 40% sodium hydroxide solution. The product, amine terminated diamino-diurea-disodium carboxylate of 17.97% in water was synthesized by this technique and abbreviated here as X2.

### Example 2—Amine, Hydroxy and Acid Functional Amino Acid Epoxide Adduct

Weigh out 516 g water into a reaction kettle. Add 100 g beta alanine under agitation at room temperature. To this solution, add 112.23 g 40% sodium hydroxide aqueous solution under agitation. Raise the temperature of this solution to 75° C. Add 136.36 g resorcinol diglycidyl ether dropwise under agitation below the temperature 85° C. Addition of this diepoxy may raise temperature to 85° C. Continue reaction for 1 hours at 85° C. Cool down to room temperature under agitation. The product, secondary amine functional diamino-diol-disodium carboxylate 30.19% in water was synthesized by this technique and abbreviated here as X3.

### Example 3—Environmentally Non-Biodegradable Controlled Release Perfume Encapsulated Particles Comprising Amino Acid Isocyanate Adducts X1, X2 and Amino Acid Epoxide Adduct X3

### Example 3A

To prepare for the active perfume phase, weigh out 125 g perfume into a 300 mL jar. To this jar, add 14 g tetraglycidyl ether sorbitol, 5 g epoxy phenol novolac triglycidyl ether, 14 g diphenyldiisocyanate based polymeric isocyanate, 5 g hexamethylene diisocyanate biuret and close the jar by its lid and agitate by magnet bar for 30 minutes. To prepare aqueous phase, weigh out 250 g distilled water into a reaction kettle. Add 0.55 g of 70% nonylphenyl ethoxylated surfactant (of HLB˜17) to this kettle and agitate. Raise the temperature of this reaction kettle to 40° C. under agitation. Once the temperature of aqueous phase in the reaction kettle is at 40° C., add active perfume phase into the kettle under agitation of 800 rpm to prepare oil-in-water (O/W) emulsion. After 15 minutes of agitation at 40° C., add dropwise 157.53 g of X1 crosslinker. Continue agitation for 15 minutes and add 0.5 g of 50% aqueous dispersion of 2,4-dimethyl imidazole. Allow the agitation at the same condition for 30 minutes. Raise the kettle temperature to 60° C. and allow the reaction to happen 3 hours at 60° C. Add 200 g distilled water and cool down kettle further to 40° C. while agitating and adjust pH to 5.5 by adding 22.65 g 20% hydrochloride aqueous solution. In the meantime, prepare aqueous solution of tetrahydroxy orthosiloxane or orthosilicic acid from tetraethylorthosiloxane (TEOS). Weigh out 65 g tetraethyl orthosilicate and 65 g of distilled water into a separate and small reaction kettle and add few drops of 20% hydrochloride aqueous solution to adjust pH to ˜2 and agitate at 45° C. for nearly about 30 minutes. Rota-vap this freshly produced orthosilicic acid at 35° C. for about 30 minutes to remove ethanol. Add 95 g of 31.58% aqueous solution of orthosilicic acid into main reaction kettle under agitation and continue agitation for 16 hours while cooling down to room temperature. Next, raise pH to 8.5 by adding 9.38 g 40% sodium hydroxide aqueous solution. Add 53.77 g 20% calcium chloride aqueous solution dropwise under agitation. Continue agitation for 15 minutes at room temperature and add 1000 g distilled water under agitation. This whole process produced filterable perfume encapsulated microcapsules. Filter precipitate with 2 μm cloth and dry the product at room temperature for 24 hours and then at 110° C. for 30 minutes. These microcapsules contain 53.28% perfume in dry powder form.

### Example 3B

To prepare for the active perfume phase, weigh out 125 g perfume into a 300 mL jar. To this jar, add 14 g tetraglycidyl ether sorbitol, 5 g epoxy phenol novolac triglycidyl ether, 14 g diphenyldiisocyanate based polymeric isocyanate, 5 g hexamethylene diisocyanate biuret and close the jar by its lid and agitate by magnet bar for 30 minutes. To prepare aqueous phase, weigh out 250 g distilled water into a reaction kettle. Add 0.55 g of 70% nonylphenyl ethoxylated surfactant (of HLB˜17) to this kettle and agitate. Raise the temperature of this reaction kettle to 40° C. under agitation. Once the temperature of aqueous phase in the reaction kettle is at 40° C. add active perfume phase into the kettle under agitation of 800 rpm to prepare oil in water emulsion. After 15 minutes of agitation at 40° C., add dropwise 274.69 g of X2 crosslinker. Continue agitation for 15 minutes and add 0.5 g of 50% aqueous dispersion of 2,4-dimethyl imidazole. Allow the agitation at the same condition for 30 minutes. Raise the kettle temperature to 60° C. and allow the reaction to happen 3 hours at 60° C. Add 200 g distilled water and cool down kettle further to 40° C. while agitating and adjust pH to 5.5 by adding 22.65 g 20% hydrochloride aqueous solution. In the meantime, prepare aqueous solution of tetrahydro orthosiloxane or orthosilicic acid from tetraethylorthosiloxane (TEOS). Weigh out 65 g tetraethyl orthosilicate and 65 g of distilled water into a separate and small reaction kettle and add few drops of 20% hydrochloride aqueous solution to adjust pH to ˜2 and agitate at 45° C. for nearly about 30 minutes. Rota-vap this freshly produced orthosilicic acid at 35° C. for about 30 minutes to remove ethanol. Add 95 g of 31.58% aqueous solution of orthosilicic acid into main reaction kettle under agitation and continue agitation for 16 hours while cooling down to room temperature. Next, raise pH to 8.5 by adding 9.38 g 40% sodium hydroxide aqueous solution. Add 53.70 g 20% calcium chloride aqueous solution dropwise under agitation. Continue agitation for 15 minutes at room temperature and add 1000 g distilled water under agitation. This whole process produced filterable perfume encapsulated microcapsules. Filter precipitate with 2 μm cloth and dry the product at room temperature for 24 hours and then at 110° C. for 30 minutes. These microcapsules contain 54.40% perfume in dry powder form.

### Example 3C

To prepare for the active perfume phase, weigh out 125 g perfume into a 300 mL jar. To this jar add 14 g tetraglycidyl ether sorbitol, 5 g epoxy phenol novolac triglycidyl ether, 14 g diphenyldiisocyanate based polymeric isocyanate, 5 g hexamethylene diisocyanate biuret and close the jar by its lid and agitate by magnet bar for 30 minutes. To prepare aqueous phase, weigh out 250 g distilled water into a reaction kettle. Add 0.55 g of 70% nonylphenyl ethoxylated surfactant (of HLB˜17) to this kettle and agitate. Raise the temperature of this reaction kettle to 40° C. under agitation. Once the temperature of aqueous phase in the reaction kettle is at 40° C. add active perfume phase into the kettle under agitation of 800 rpm to prepare oil in water emulsion. After 15 minutes of agitation at 40° C., add dropwise a combined solution of 78.76 g X1 and 100.74 g X3 crosslinkers. Continue agitation for 15 minutes and add 0.5 g of 50% aqueous dispersion of 2,4-dimethyl imidazole. Allow the agitation at the same condition for 30 minutes. Raise the kettle temperature to 60° C. and allow the reaction to happen 3 hours at 60° C. Add 200 g distilled water and cool down kettle further to 40° C. while agitating and adjust pH to 5.5 by adding 26.26 g 20% hydrochloride aqueous solution. In the meantime, prepare aqueous solution of tetrahydroxy orthosiloxane or orthosilicic acid from tetraethylorthosiloxane (TEOS). Weigh out 65 g tetraethyl orthosilicate and 65 g of distilled water into a separate and small reaction kettle and add few drops of 20% hydrochloride aqueous solution to adjust pH to ˜2 and agitate at 45° C. for nearly about 30 minutes. Rota-vap this freshly produced orthosilicic acid at 35° C. for about 30 minutes to remove ethanol. Add 95 g of 31.58% aqueous solution of orthosilicic acid into main reaction kettle under agitation and d continue agitation for 16 hours while cooling down to room temperature. Next, raise pH to 8.5 by adding 13.86 g 40% sodium hydroxide aqueous solution. Add 63.26 g 20% calcium chloride aqueous solution dropwise under agitation. Continue agitation for 15 minutes at room temperature and add 1000 g distilled water under agitation. This whole process produced filterable perfume encapsulated microcapsules. Filter precipitate with 2 μm cloth and dry the product at room temperature for 24 hours and then at 110° C. for 30 minutes. These microcapsules contain 52.55% perfume in dry powder form.

### Example 3D

To prepare for the active perfume phase, weigh out 125 g perfume into a 300 mL jar. To this jar add 14 g tetraglycidyl ether sorbitol, 5 g epoxy phenol novolac triglycidyl ether, 14 g diphenyldiisocyanate based polymeric isocyanate, 5 g hexamethylene diisocyanate biuret and close the jar by its lid and agitate by magnet bar for 30 minutes. To prepare aqueous phase, weigh out 250 g distilled water into a reaction kettle. Add 0.55 g of 70% nonylphenyl ethoxylated surfactant (of HLB˜17) to this kettle and agitate. Raise the temperature of this reaction kettle to 40° C. under agitation. Once the temperature of aqueous phase in the reaction kettle is at 40° C. add active perfume phase into the kettle under agitation of 800 rpm to prepare oil in water emulsion. After 15 minutes of agitation at 40° C., add dropwise a combined solution of 137.35 g X2 and 100.74 g X3 crosslinkers. Continue agitation for 15 minutes and add 0.5 g of 50% aqueous dispersion of 2,4-dimethyl imidazole. Allow the agitation at the same condition for 30 minutes. Raise the kettle temperature to 60° C. and allow the reaction to happen 3 hours at 60° C. Add 200 g distilled water and cool down kettle further to 40° C. while agitating and adjust pH to 5.5 by adding 28.05 g 20% hydrochloride aqueous solution. In the meantime, prepare aqueous solution of tetrahydroxy orthosiloxane or orthosilicic acid from tetraethylorthosiloxane (TEOS). Weigh out 65 g tetraethyl orthosilicate and 65 g of distilled water into a separate and small reaction kettle and add few drops of 20% hydrochloride aqueous solution to adjust pH to ˜2 and agitate at 45° C. for nearly about 30 minutes. Rota-vap this freshly produced orthosilicic acid at 35° C. for about 30 minutes to remove ethanol. Add 95 g of 31.58% aqueous solution of orthosilicic acid into main reaction kettle under agitation and d continue agitation for 16 hours while cooling down to room temperature. Next, raise pH to 8.5 by adding 12.50 g 40% sodium hydroxide aqueous solution. Add 63.22 g 20% calcium chloride aqueous solution dropwise under agitation. Continue agitation for 15 minutes at room temperature and add 1000 g distilled water under agitation. This whole process produced filterable perfume encapsulated microcapsules. Filter precipitate with 2 μm cloth and dry the product at room temperature for 24 hours and then at 110° C. for 30 minutes. These microcapsules contain 53.68% perfume in dry powder form.

### Example 3E

To prepare for the active perfume phase, weigh out 125 g perfume into a 300 mL jar. To this jar add 14 g tetraglycidyl ether sorbitol, 5 g epoxy phenol novolac triglycidyl ether, 14 g diphenyldiisocyanate based polymeric isocyanate, 5 g hexamethylene diisocyanate biuret and close the jar by its lid and agitate by magnet bar for 30 minutes. To prepare aqueous phase, weigh out 250 g distilled water into a reaction kettle. Add 0.55 g of 70% nonylphenyl ethoxylated surfactant (of HLB˜17) to this kettle and agitate. Raise the temperature of this reaction kettle to 40° C. under agitation. Once the temperature of aqueous phase in the reaction kettle is at 40° C. add active perfume phase into the kettle under agitation of 800 rpm to prepare oil in water emulsion. After 15 minutes of agitation at 40° C., add dropwise a combined solution of 53.06 g X1, 92.53 g X2, and 67.86 g X3 crosslinkers. Continue agitation for 15 minutes and add 0.5 g of 50% aqueous dispersion of 2,4-dimethyl imidazole. Allow the agitation at the same condition for 30 minutes. Raise the kettle temperature to 60° C. and allow the reaction to happen 3 hours at 60° C. Add 200 g distilled water and cool down kettle further to 40° C. while agitating and adjust pH to 5.5 by adding 28.15 g 20% hydrochloride aqueous solution. In the meantime, prepare aqueous solution of tetrahydroxy orthosiloxane or orthosilicic acid from tetraethylorthosiloxane (TEOS). Weigh out 65 g tetraethyl orthosilicate and 65 g of distilled water into a separate and small reaction kettle and add few drops of 20% hydrochloride aqueous solution to adjust pH to ˜2 and agitate at 45° C. for nearly about 30 minutes. Rota-vap this freshly produced orthosilicic acid at 35° C. for about 30 minutes to remove ethanol. Add 95 g of 31.58% aqueous solution of orthosilicic acid into main reaction kettle under agitation and d continue agitation for 16 hours while cooling down to room temperature. Next, raise pH to 8.5 by adding 12.50 g 40% sodium hydroxide aqueous solution. Add 60.71 g 20% calcium chloride aqueous solution dropwise under agitation. Continue agitation for 15 minutes at room temperature and add 1000 g distilled water under agitation. This whole process produced filterable perfume encapsulated microcapsules. Filter precipitate with 2 μm cloth and dry the product at room temperature for 24 hours and then at 110° C. for 30 minutes. These microcapsules contain 53.64% perfume in dry powder form.

### Example 3F

To prepare for the active perfume phase, weigh out 125 g perfume into a 300 mL jar. To this jar add 14 g tetraglycidyl ether sorbitol, 5 g epoxy phenol novolac triglycidyl ether, 14 g diphenyldiisocyanate based polymeric isocyanate, 5 g hexamethylene diisocyanate biuret, 2.0 g plasticizer and close the jar by its lid and agitate by magnet bar for 30 minutes. Add 2.0 g fine sorbitol and mix for 1 minute at 10000 rpm. To prepare aqueous phase, weigh out 250 g distilled water into a reaction kettle. Add 0.55 g of 70% nonylphenyl ethoxylated surfactant (of HLB˜17) to this kettle and agitate. Raise the temperature of this reaction kettle to 40° C. under agitation. Once the temperature of aqueous phase in the reaction kettle is at 40° C. add active perfume phase into the kettle under agitation of 800 rpm to prepare oil in water emulsion. After 15 minutes of agitation at 40° C., add dropwise a combined solution of 53.06 g X1, 92.53 g X2, and 67.86 g X3 crosslinkers. Continue agitation for 15 minutes and add 0.5 g of 50% aqueous dispersion of 2,4-dimethyl imidazole. Allow the agitation at the same condition for 30 minutes. Raise the kettle temperature to 60° C. and allow the reaction to happen 3 hours at 60° C. Add 200 g distilled water and cool down kettle further to 40° C. while agitating and adjust pH to 5.5 by adding 28.15 g 20% hydrochloride aqueous solution. In the meantime, prepare aqueous solution of tetrahydroxy orthosiloxane or orthosilicic acid from tetraethylorthosiloxane (TEOS). Weigh out 65 g tetraethyl orthosilicate and 65 g of distilled water into a separate and small reaction kettle and add few drops of 20% hydrochloride aqueous solution to adjust pH to ˜2 and agitate at 45° C. for nearly about 30 minutes. Rota-vap this freshly produced orthosilicic acid at 35° C. for about 30 minutes to remove ethanol. Add 95 g of 31.58% aqueous solution of orthosilicic acid into main reaction kettle under agitation and d continue agitation for 16 hours while cooling down to room temperature. Next, raise pH to 8.5 by adding 12.50 g 40% sodium hydroxide aqueous solution. Add 25.0 g 20% calcium chloride aqueous solution dropwise under agitation. Continue agitation for 15 minutes at room temperature and add 500 g distilled water under agitation. This whole process produced optional filterable perfume encapsulated microcapsules. Filter precipitate with 2 μm cloth and dry the product at room temperature for 24 hours and then at 110° C. for 30 minutes. These microcapsules contain 53.24% perfume in dry powder form.

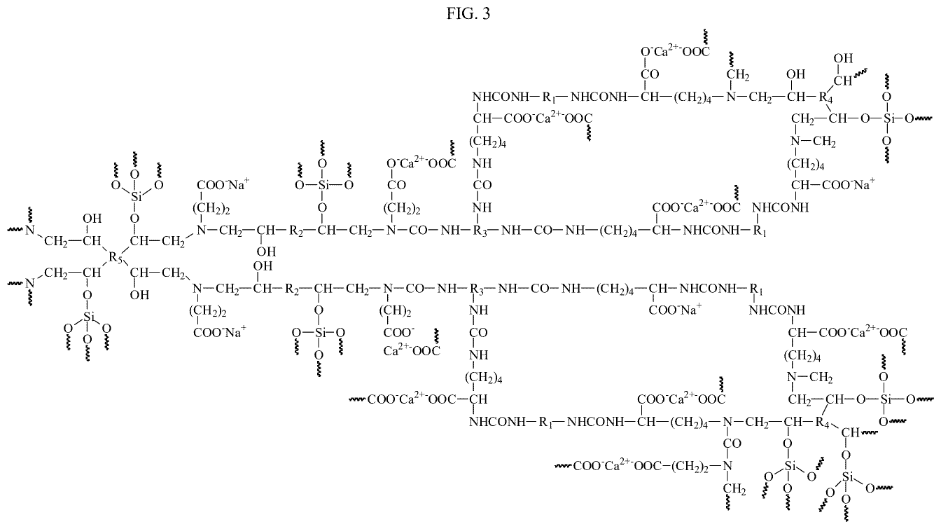

A common and representative microcapsule shell of crosslinked polymer as formed as per example 3A-3F is depicted in FIG. 3. R₃ is a moiety of monomer or prepolymer holding three reactive-NCO functional groups; R₄ is a moiety of monomer or prepolymer having three reactive glycidyl ether epoxide groups; R₅ is a moiety of a monomer or prepolymer having four reactive glycidyl ether epoxide groups.

### Example 4 Agricultural Active Encapsulation

### Example 4A

To prepare aqueous phase, weigh out water 200 g water into a reaction kettle. Add 11 g whey protein under agitation. Raise temperature of water phase in the kettle to 65° C. Add 20% sodium metasilicate to adjust pH to 6.5-7.0. Add 5 g 70% nonylphenyl ethoxylated surfactant (of HLB˜18). To prepare agricultural active containing organic phase, weigh out 200 g N-(1-ethylpropyl)-3,4-dimethyl-2,6-dinitrobenzenamine and melt at 60-75° C. under mild agitation. Add 3.75 g diphenyldiisocyanate based polymeric isocyanate and 4.5 g trimethylolpropane-xylenediisocyanate adduct based triisocyanate and agitate at 60-75° C. for ˜ 1 minute to solubilize isocyanates in the active material. Add organic phase (kept at 60-75° C.) into aqueous phase (kept at 65° C.) under 2000-3000 rpm. Continue agitation at 65° C. for 2.5 minutes, adjust pH to 8.5 by adding 20% sodium metasilicate. After 5 minutes, add 10 g X1 of Example 1A and 10 g X3 of Example 2 blend dropwise. Continue agitation at 65° C. for 2 hours. Cool down to room temperature while agitating the slurry. Add 5 g sodium lignosulfonate, and 50.2 g aqueous solution having 20 g magnesium sulfate and 0.2 g gum arabic. Adjust pH of the slurry to 7.5 . . . . Accounting for the loss of water during processing, the actual active content in the slurry is 42.37%.

### Example 4B

To prepare aqueous phase, weigh out water 200 g water into a reaction kettle. Add 11 g whey protein under agitation. Raise temperature of water phase in the kettle to 65° C. Add 20% sodium metasilicate to adjust pH to 6.5-7.0. Add 5 g 70% nonylphenyl ethoxylated surfactant (of HLB˜18). Add 5 g sodium lignosulfonate. To prepare agricultural active containing organic phase, weigh out 200 g N-(1-ethylpropyl)-3,4-dimethyl-2,6-dinitrobenzenamine and melt at 60-75° C. under mild agitation. Add 3.75 g diphenyldiisocyanate based polymeric isocyanate and 4.5 g trimethylolpropane-xylenediisocyanate adduct based triisocyanate and agitate at 60-75 C for ˜ 1 minute to solubilize isocyanates in active material. Add organic phase (kept at 60-75° C.) into aqueous phase (kept at 65° C.) under 2000-3000 rpm. Continue agitation at 65° C. for 2.5 minutes, adjust pH to 8.5 by adding 20% sodium metasilicate. After 5 minutes, add 10 g X1 of Example 1A and 10 g X3 of Example 2 blend dropwise. Continue agitation at 65° C. for 2 hours. Cool down to room temperature while agitating the slurry. Add 2 g sodium naphthalene sulfonate and 50.2 g aqueous solution having 20 g magnesium sulfate and 0.2 g xanthan gum. At room temperature adjust pH of the slurry to 7.5 . . . . Accounting for the loss of water during manufacturing, the actual active content in the slurry is 42.19%.

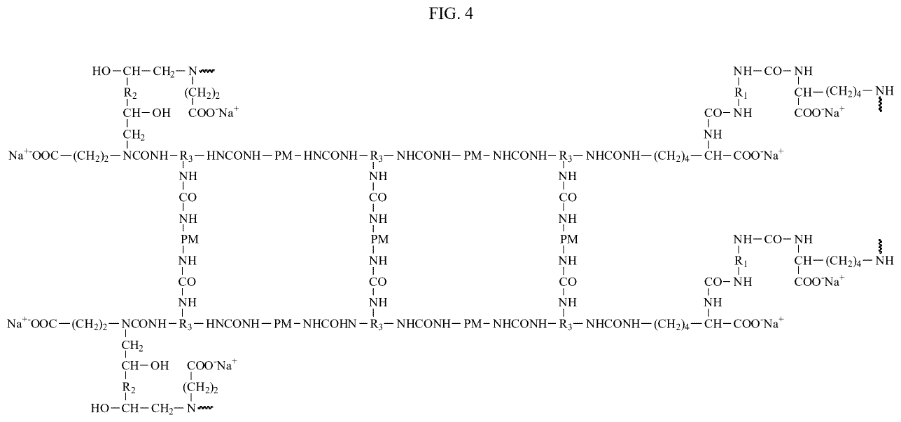

A common and representative microcapsule shell of crosslinked polymer as formed as per example 4A-4B is depicted in FIG. 4. R₃ is a moiety of monomer or prepolymer holding three reactive-NCO functional groups; R₄ is a moiety of monomer or prepolymer having three reactive glycidyl ether epoxide groups; R₅ is a moiety of a monomer or prepolymer having four reactive glycidyl ether epoxide groups; and PM is a moiety of a protein or a peptide.

### Example 5—Leakage Stability

Microcapsule leakage is measured using the Leakage Method.

TABLE 4A
Leakage of perfume oil from capsules after
exposure to 1% uric acid solution.

| | | % Leakage in 1% Uric |
|---|---|---|
| ID | Description of Capsule | Acid Solution |
| Example 5A | Example 3B Capsules | 63% |
| Example 5B | Example 3C Capsules | 80% |
| Example 5C | Example 3E Capsules | 65% |

### Example 6—Bleach Tablet

Table 6.1 and 6.2 below list common ingredients in bleach tablet compositions for a toilet bowl cleaner. It should be noted that the tables represent possible embodiments and compositions of the cleaner and that the cleaner is in no way limited to these exact compositions.

TABLE 6.1

| Ingredient | Parts |
|---|---|
| Citric Acid | 31 |
| Sodium Bicarbonate | 49 |
| Polyethylene Glycol 8000 | 5 |
| Sorbitol | 10 |
| CDB | 2 |
| Sodium Benzoate | 2 |
| Spray Dried Microcapsules of Example 3 | 0.5 |
| Bioterget | 0.2 |
| Rhodasurf | 0.1 |

TABLE 6.2

| Ingredient | Parts |
|---|---|
| Citric Acid | 31 |
| Sodium Bicarbonate | 50 |
| Polyethylene Glycol 8000 | 5 |
| Sorbitol | 9.3 |
| Sodium Perborate | 1 |
| Sodium Benzoate | 2 |
| Spray Dried Microcapsules of Example 3 | 0.5 |
| Copper sulfate | 0.5 |
| bicinchoninic acid | 0.2 |
| Bioterge | 0.2 |
| Rhodasurf | 0.1 |

All of the ingredients (except the sodium benzoate) are added into a low shear powder blender and mixed for 20 minutes. Sodum benzoate is then added and mixed for 3 minutes, pressed into tablets, and packaged in moisture protected packaging. All of the mixed ingredients could optionally be granulated instead of tableted. Granulation can be achieved by passing the mixed powder through a roll compactor, then gently grinding the powder and classifying the powder to collect the desired particle size range.

### Example 7—Hair Conditioner

Selected microcapsules from the above examples are formulated into a leave-on-conditioner formulation as follows: to 98.0 grams of leave-on-conditioner (with a typical formulation given below) is added an appropriate amount of microcapsule slurry of the above examples, to deliver an encapsulated oil usage level of 0.5 wt. %. The microcapsules are added on top of the conditioner formulation, then the contents are mixed at 1000 RPM for 1 minute.

A typical composition of a leave-on conditioner formulation is given in Table 6.1 below.

TABLE 7.1
Hair Condition Formulation

| Components | Ex. I (LOT) (%) |
|---|---|
| Premix | |
| Aminosilicone | — |
| PDMS | 1.0-1.5 |
| Gel matrix carrier | |
| Behenyl trimethyl ammonium chloride | — |
| Stearamidopropyldimethylamine | 0.60-0.8 |
| (SAPDMA), C18 | |
| DTDMAC, C18(Quaternium-18) | 0.45-0.6 |
| Citric Acid (anhydrous) | 0.10-0.25 |
| Cetyl alcohol | 0.80-1.0 |
| Stearyl alcohol | 0.54-1.0 |
| Deionized Water | Balance |
| Polymers | |
| Hydroxyethylcellulose (HEC) | 0.15-0.50 |
| PEG-2M (Polyox WAR N-10) | 0.30-0.60 |
| Others | |
| Preservatives | 0.40-0.60 |

### Example 8—Shampoo

Selected microcapsules from the above examples are formulated into a rinse-off shampoo formulation as follows: to 90.0 grams of shampoo formulation is added an appropriate amount of microcapsule slurry of Example 3, to deliver an encapsulated oil usage level of 0.5 wt. %. The microcapsules and water are added on top of the shampoo formulation, then the contents are mixed at 1850 RPM for 1 minute. Typical shampoo formulations are shown in Tables 8.1, 8.2 and 8.3 below.

TABLE 8.1
Shampoo Formulations of Examples 7A-7C.

| | Example | | |
|---|---|---|---|
| Ingredient | 8A | 8B | 8C |
| Water | q.s. | q.s. | q.s. |
| Polyquaternium 76 ¹ | 2.50 | — | — |
| Guar, Hydroxylpropyl Trimonium | — | 0.25 | — |
| Chloride ² | | | |
| Polyquaterium 6 ³ | — | — | 0.79 |
| Sodium Laureth Sulfate (SLE3S) ⁴ | 21.43 | 21.43 | 21.43 |
| Sodium Lauryl Sulfate (SLS) ⁵ | 20.69 | 20.69 | 20.69 |
| Silicone ⁶ | 0.75 | 1.00 | 0.5 |
| Cocoamidopropyl Betaine ⁷ | 3.33 | 3.33 | 3.33 |
| Cocoamide MEA ⁸ | 1.0 | 1.0 | 1.0 |
| Ethylene Glycol Distearate ⁹ | 1.50 | 1.50 | 1.50 |
| Sodium Chloride ¹⁰ | 0.25 | 0.25 | 0.25 |
| Fragrance | 0.70 | 0.70 | 0.70 |
| Fragrance Microcapsules | 1.2 | 1.2 | 1.2 |
| Preservatives, pH adjusters | Up to 1% | Up to 1% | Up to 1% |

¹ Mirapol AT-1, Copolymer of Acrylamide(AM) and TRIQUAT, MW = 1,000,000; CD = 1.6 meq./gram; 10% active; Supplier Rhodia
² Jaguar C500, MW - 500,000, CD = 0.7, supplier Rhodia
³ Mirapol 100S, 31.5% active, supplier Rhodia
⁴ Sodium Laureth Sulfate, 28% active, supplier: P&G
⁵ Sodium Lauryl Sulfate, 29% active supplier: P&G
⁶ Glycidol Silicone VC2231-193C
⁷ Tegobetaine F-B, 30% active supplier: Goldschmidt Chemicals
⁸ Monamid CMA, 85% active , supplier Goldschmidt Chemical
⁹ Ethylene Glycol Distearate, EGDS Pure, supplier Goldschmidt Chemical
¹⁰ Sodium Chloride USP (food grade), supplier Morton; note that salt is an adjustable ingredient, higher or lower levels may be added to achieve target viscosity.

TABLE 8.2
Shampoo Formulations of Examples 8D-8F.

| | Example | | |
|---|---|---|---|
| Ingredient | 8D | 8E | 8F |
| Water | q.s. | q.s. | q.s. |
| Silicone A ¹ | 1.0 | 0.5 | 0.5 |
| Cyclopentasiloxane ⁴ | — | 0.61 | 1.5 |
| Behenyl trimethyl ammonium chloride ⁵ | 2.25 | 2.25 | 2.25 |
| Isopropyl alcohol | 0.60 | 0.60 | 0.60 |
| Cetyl alcohol ⁶ | 1.86 | 1.86 | 1.86 |
| Stearyl alcohol ⁷ | 4.64 | 4.64 | 4.64 |
| Disodium EDTA | 0.13 | 0.13 | 0.13 |
| NaOH | 0.01 | 0.01 | 0.01 |
| Benzyl alcohol | 0.40 | 0.40 | 0.40 |
| Methylchloroisothiazolinone/ | 0.0005 | 0.0005 | 0.0005 |
| Methylisothiazolinone ⁸ | | | |
| Panthenol ⁹ | 0.10 | 0.10 | 0.10 |
| Panthenyl ethyl ether ¹⁰ | 0.05 | 0.05 | 0.05 |
| Fragrance | 0.35 | 0.35 | 0.35 |
| Fragrance Microcapsules | 1.2 | 1.2 | 1.2 |

¹ Glycidol Silicone
⁴ Cyclopentasiloxane: SF1202 available from Momentive Performance Chemicals
⁵ Behenyl trimethyl ammonium chloride/Isopropyl alcohol: Genamin TM KMP available from Clariant
⁶ Cetyl alcohol: Konol TM series available from Shin Nihon Rika
⁷ Stearyl alcohol: Konol TM series available from Shin Nihon Rika
⁸ Methylchloroisothiazolinone/Methylisothiazolinone: Kathon TM CG available from Rohm & Haas
⁹ Panthenol: Available from Roche
¹⁰ Panthenyl ethyl ether: Available from Roche

TABLE 8.3
Shampoo Formulations of Examples 8G and 8H

| | Example | |
|---|---|---|
| Ingredient | 8G | 8H |
| Sodium Laureth Sulfate | 10.00 | 10.00 |
| Sodium Lauryl Sulfate | 1.50 | 1.50 |
| Cocamidopropyl betaine | 2.00 | 2.00 |
| Guar Hydroxypropyl trimonium chloride ¹ | 0.40 | |
| Guar Hydroxypropyl trimonium chloride ² | | 0.40 |
| Dimethicone ³ | 2.00 | 2.00 |
| Gel Network ⁴ | | 27.27 |
| Ethylene Glycol Distearate | 1.50 | 1.50 |
| 5-Chloro-2-methyl-4-isothiazolin-3-one, | 0.0005 | 0.0005 |
| Kathon CG | | |
| Sodium Benzoate | 0.25 | 0.25 |
| Disodium EDTA | 0.13 | 0.13 |
| Perfume | 0.40 | 0.40 |
| Fragrance Microcapsules | 0.30 | 0.30 |
| Citric Acid/Sodium Citrate Dihydrate | pH QS | pH QS |
| Sodium Chloride/Ammonium Xylene | Visc. QS | Visc. QS |
| Sulfonate | | |
| Water | QS | QS |

¹ Jaguar C17 available from Rhodia
² N-Hance 3269 (with Mol. W. of ~500,000 and 0.8 meq/g) available from Aqulaon/Hercules
³ Viscasil 330M available from General Electric Silicones
⁴ Gel Networks; See composition in Table 7.4 below. The water is heated to about 74° C. and the Cetyl Alcohol, Stearyl Alcohol, and the SLES Surfactant are added to it. After incorporation, this mixture is passed through a heat exchanger where it is cooled to about 35° C. As a result of this cooling step, the Fatty Alcohols and surfactant crystallized to form a crystalline gel network.

TABLE 8.4
Gel Network Composition

| Ingredient | Wt. % |
|---|---|
| Water | 86.14% |
| Cetyl Alcohol | 3.46% |
| Stearyl Alcohol | 6.44% |
| Sodium laureth-3 sulfate (28% Active) | 3.93% |
| 5-Chloro-2-methyl-4-isothiazolin-3-one, Kathon CG | 0.03% |

### Example 9—Lotion

For the examples shown in Table 8 below, in a suitable container, combine the ingredients of Phase A. In a separate suitable container, combine the ingredients of Phase B. Heat each phase to 73° C.-78° C. while mixing each phase using a suitable mixer (e.g., Anchor blade, propeller blade, or IKA T25) until each reaches a substantially constant desired temperature and is homogenous. Slowly add Phase B to Phase A while continuing to mix Phase A. Continue mixing until batch is uniform. Pour product into suitable containers at 73-78° C. and store at room temperature. Alternatively, continuing to stir the mixture as temperature decreases results in lower observed hardness values at 21 and 33° C.

TABLE 9
Lotion Formulations (Examples 9A-9C).

| | Example | | |
|---|---|---|---|
| Ingredient/Property | 9A | 9B | 9C |
| PHASE A | | | |
| DC-9040 ¹ | 8.60 | 3.00 | 5.00 |
| Dimethicone | 4.09 | 4.00 | 4.00 |
| Polymethylsilsesquioxane ² | 4.09 | 4.00 | 4.00 |
| Cyclomethicone | 11.43 | 0.50 | 11.33 |
| KSG-210 ³ | 5.37 | 5.25 | 5.40 |
| Polyethylene wax ⁴ | 3.54 | | 2.05 |
| DC-2503 Cosmetic Wax ⁵ | 7.08 | 10.00 | 3.77 |
| Hydrophobic TiO₂ | | | 0.50 |
| Iron oxide coated Mica | | | 0.65 |
| TiO₂ Coated Mica | 1.00 | 1.00 | |
| Fragrance Microcapsules | 1.00 | 1.00 | 1.00 |
| PHASE B | | | |
| Glycerin | 10.00 | 10.00 | 10.00 |
| Dexpanthenol | 0.50 | 0.50 | 0.50 |
| Pentylene Glycol | 3.00 | 3.00 | 3.00 |
| Hexamidine Diisethionate ⁶ | 0.10 | 0.10 | 0.10 |
| Niacinamide ⁷ | 5.00 | 5.00 | 5.00 |
| Methylparaben | 0.20 | 0.20 | 0.20 |
| Ethylparaben | 0.05 | 0.05 | 0.05 |
| Sodium Citrate | 0.20 | 0.20 | 0.20 |
| Citric Acid | 0.03 | 0.03 | 0.03 |
| Sodium Benzoate | 0.05 | 0.05 | 0.05 |
| Sodium Chloride | 0.50 | 0.50 | 0.50 |
| FD&C Red #40 (1%) | 0.05 | 0.05 | 0.05 |
| Water | q.s to 100 | q.s to 100 | q.s to 100 |
| Hardness at 21° C. (g) | 33.3 | 15.4 | 14.2 |
| Hardness at 33° C. (g) | 6.4 | 0.7 | 4.0 |

¹ 12.5% Dimethicone Crosspolymer in Cyclopentasiloxane. Available from Dow Corning.
² E.g., TOSPEAR 145A or TOSPEARL 2000. Available from GE Toshiba Silicon.
³ 25% Dimethicone PEG-10/15 Crosspolymer in Dimethicone. Available from Shin-Etsu.
⁴ JEENATE 3H polyethylene wax from Jeen.
⁵ Stearyl Dimethicone. Available from Dow Corning.
⁶ Hexamidine diisethionate, available from Laboratoires Serobiologiques.
⁷ Additionally or alternatively, the composition may comprise one or more other skin care actives, their salts and derivatives, as disclosed herein, in amounts also disclosed herein as would be deemed suitable by one of skill in the art.

### Example 10—Antiperspirant/Deodorant

Example 10A of Table 10.1 below can be made via the following general process, which one skilled in the art will be able to alter to incorporate available equipment. The ingredients of Part I and Part II are mixed in separate suitable containers. Part II is then added slowly to Part I under agitation to assure the making of a water-in-silicone emulsion. The emulsion is then milled with a suitable mill, for example a Greeco 1L03 from Greeco Corp, to create a homogenous emulsion. Part III is mixed and heated to 88° C. until the all solids are completely melted. The emulsion is then also heated to 88° C. and then added to the Part 3 ingredients. The final mixture is then poured into an appropriate container, and allowed to solidify and cool to ambient temperature.

TABLE 10.1
Antiperspirant/Deodorant Formulation (Example 10A).

| Ingredient | Example 10A |
|---|---|
| Part I: Partial Continuous Phase | |
| Hexamethyldisiloxane¹ | QS |
| DC5200² | 1.20 |
| Fragrance | 0.35 |
| Fragrance Capsules | 1.00 |
| Part II: Disperse Phase | |
| ACH (40% solution)³ | 40.00 |
| propylene glycol | 5.00 |
| Water | 12.30 |
| Part III: Structurant Plus Remainder of Continuous Phase | |
| FINSOLVE TN | 6.50 |

QS—indicates that this material is used to bring the total to 100%.
¹DC 246 fluid from Dow Corning
²from Dow Corning
³Standard aluminum chlorohydrate solution

Examples 10B to 10E of Table 10.2 below can be made as follows: all ingredients except the fragrance, and fragrance capsules are combined in a suitable container and heated to about 85° C. to form a homogenous liquid. The solution is then cooled to about 62° C. and then the fragrance, and fragrance microcapsules are added. The mixture is then poured into an appropriate container and allowed to solidify up cooling to ambient temperature.

Example 10F of Table 10.2 can be made as follows: all the ingredients except the propellant are combined in an appropriate aerosol container. The container is then sealed with an appropriate aerosol delivery valve. Next air in the container is removed by applying a vacuum to the valve and then propellant is added to container through the valve. Finally an appropriate actuator is connected to the valve to allow dispensing of the product.

TABLE 10.2
Antiperspirant/Deodorant Formulations

| | Example | | | | |
|---|---|---|---|---|---|
| Ingredient | 10B | 10C | 10D | 10E | 10F |
| Product Form | Solid | Solid | Solid | Solid | Deodorant |
| | De- | De- | De- | De- | or Body |
| | odorant | odorant | odorant | odorant | Spray |
| dipropylene glycol | 45 | 22 | 20 | 30 | 20 |
| propylene glycol | 22 | 45 | 22 | | |
| tripopylene glycol | | | 25 | | |
| Glycerine | | | | 10 | |
| PEG -8 | | | | 20 | |
| ethanol | | | | | QS |
| Water | QS | QS | QS | QS | |
| sodium stearate | 5.5 | 5.5 | 5.5 | 5.5 | |
| tetra sodium EDTA | 0.05 | 0.05 | 0.05 | 0.05 | |
| sodium hydroxide | 0.04 | 0.04 | 0.04 | 0.04 | |
| triclosan | 0.3 | 0.3 | 0.3 | 0.3 | |
| Fragrance | 0.5 | 0.5 | 0.5 | 0.5 | 0.5 |
| Fragrance capsules | 1.0 | 1.0 | 1.0 | 1.0 | 0.5 |
| Propellant (1,1 | | | | | 40 |
| difluoroethane) | | | | | |

QS—indicates that this material is used to bring the total to 100%.

### Example 11—Rinse-off Conditioner

The conditioning compositions of Examples 11A through 11F of Table 11 are prepared as follows: cationic surfactants, high melting point fatty compounds are added to water with agitation, and heated to about 80° C. The mixture is cooled down to about 50° C. to form a gel matrix carrier. Separately, slurries of perfume microcapsules and silicones are mixed with agitation at room temperature to form a premix. The premix is added to the gel matrix carrier with agitation. If included, other ingredients such as preservatives are added with agitation. Then the compositions are cooled down to room temperature.

The conditioning composition of Example 11B of Table 11 is prepared as follows: cationic surfactants, high melting point fatty compounds are added to water with agitation, and heated to about 80° C. The mixture is cooled down to about 50° C. to form a gel matrix carrier. Then, silicones are added with agitation. Separately, slurries of perfume microcapsules, and if included, other ingredients such as preservatives are added with agitation. Then the compositions are cooled down to room temperature.

TABLE 11
Rinse-Off Conditioner Formulations (Examples 11A-11F).

| | Example | | | | | |
|---|---|---|---|---|---|---|
| Ingredient | 11A | 11B | 11C | 11D | 11E | 11F³ |
| Premix | | | | | | |
| Aminosilicone-1¹ | 0.50 | 0.50 | | | | |
| Aminosilicone-2 ² | | | 0.50 | 0.50 | 0.50 | |
| PDMS | | | | | | 0.50 |
| Fragrance microcapsules | . . . | 1.0 | 1.0 | 1.0 | 1.0 | 1.0 |
| Gel matrix carrier | | | | | | |
| Behenyl trimethyl ammonium | 2.30 | 2.30 | 2.30 | 2.30 | 2.30 | 2.30 |
| chloride | | | | | | |
| Cetyl alcohol | 1.5 | 1.5 | 1.5 | 1.5 | 1.5 | 1.5 |
| Stearyl alcohol | 3.8 | 3.8 | 3.8 | 3.8 | 3.8 | 3.8 |
| Deionized Water | QS | QS | QS | QS | QS | QS |
| Preservatives | 0.4 | 0.4 | 0.4 | 0.4 | 0.4 | 0.4 |
| Panthenol | — | — | 0.03 | — | — | — |
| Panthenyl ethyl ether | — | — | 0.03 | — | — | — |

¹Aminosilicone-1 (AMD): having an amine content of 0.12-0.15 m mol/g and a viscosity of 3,000-8,000 mPa · s, which is water insoluble
² Aminosilicone-2 (TAS): having an amine content of 0.04-0.06 m mol/g and a viscosity of 10,000-16,000 mPa · s, which is water insoluble
³Comparative example with PDMS instead of amino silicone

### Example 12—Body Cleansing Composition

The body cleaning compositions of Examples 12A-12C are prepared as follows.

The cleansing phase composition is prepared by adding surfactants, guars, and Stabylen 30 to water. Sodium chloride is then added to the mixture to thicken the cleansing phase composition. Preservatives and chelants are added to the formulation. Finally, perfume is added to the suspension.

The benefit phase composition is prepared by mixing petrolatum and mineral oil to make a homogeneous mixture. Fragrance microcapsules are added to the suspension. Finally, the cleansing phase (e.g. surfactant phase) and benefit phase are mixed in different ratios to yield the body cleansing composition.

TABLE 12
Body Cleansing Composition Formulations (Examples 12A-12C).

| | Example | | |
|---|---|---|---|
| Ingredient | 12A | 12B | 12C |
| I: Cleansing Phase Composition | | | |
| Sodium Trideceth Sulfate | 5.9 | 5.9 | 5.9 |
| (sulfated from Iconol TDA-3 | | | |
| (BASF Corp.) to >95% sulfate) | | | |
| Sodium Lauryl Sulfate | 5.9 | 5.9 | 5.9 |
| Sodium Lauroamphoacetate | 3.6 | 3.6 | 3.6 |
| (Cognis Chemical Corp.,) | | | |
| Guar Hydroxypropyltrimonium | — | 0.3 | 0.7 |
| Chloride | | | |
| (N-Hance 3196 from Aqualon) | | | |
| Guar Hydroxypropyltrimonium | 0.6 | — | — |
| Chloride | | | |
| (Jaguar C-17 from Rhodia) | | | |
| Stabylen 30 | 0.33 | 0.33 | 0.33 |
| (Acrylates/Vinyl Isodecanoate, 3V) | | | |
| Sodium Chloride | 3.75 | 3.75 | 3.75 |
| Trideceth-3 | 1.75 | 1.75 | 1.75 |
| (Iconal TDA-3 from BASF Corp.) | | | |
| Methyl chloro isothiazolinone and | 0.033 | 0.033 | 0.033 |
| methyl isothiazolinone (Kathon CG, | | | |
| Rohm & Haas) | | | |
| EDTA (Dissolvine NA 2x) | 0.15 | 0.15 | 0.15 |
| Sodium Benzoate | 0.2 | 0.2 | 0.2 |
| Citric Acid, titrate | pH = | pH = | pH = |
| | 5.7 ± 0.2 | 5.7 ± 0.2 | 5.7 ± 0.2 |
| Perfume | 1.11% | 1.11% | 1.11% |
| Water and Minors (NaOH) | Q.S. | Q.S. | Q.S. |
| II: Benefit Phase Composition | | | |
| Petrolatum | 60 | 60 | 60 |
| (G2218 from Sonnerbonn) | | | |
| Mineral Oil | 20 | 20 | 20 |
| (Hydrobrite 1000 from Sonnerbonn) | | | |
| Fragrance Microcapsules | 10 | 10 | 10 |
| III: Surfactant Phase:Benefit | 50:50 | 90:10 | 90:10 |
| Phase Blending Ratio | | | |

### Example 13—Fabric Softening Product

Non-limiting examples of product formulations containing purified perfume microcapsules of the aforementioned examples are summarized in the following table.

TABLE 13
Fabric Softening Product Formulations (Examples 13A-13J).

| | Example | | | | | | | | | |
|---|---|---|---|---|---|---|---|---|---|---|
| Ingredient | 13A | 13B | 13C | 13D | 13E | 13F | 13G | 13H | 13I | 13J |
| FSA ^{a} | 14 | 16.47 | 14 | 12 | 12 | 16.47 | 3.00 | 6.5 | 5 | 5 |
| Ethanol | 2.18 | 2.57 | 2.18 | 1.95 | 1.95 | 2.57 | — | — | 0.81 | 0.81 |
| Isopropyl | — | — | — | — | — | — | 0.33 | 1.22 | — | — |
| Alcohol | | | | | | | | | | |
| Microcapsule | 0.6 | 0.75 | 0.6 | 0.75 | 0.37 | 0.60 | 0.37 | 0.6 | 0.37 | 0.37 |
| (% active)* | | | | | | | | | | |
| Phase | 0.21 | 0.25 | 0.21 | 0.21 | 0.14 | — | — | 0.14 | — | — |
| Stabilizing | | | | | | | | | | |
| Polymer ^{f} | | | | | | | | | | |
| Suds | — | — | — | — | — | — | — | 0.1 | — | — |
| Suppressor ^{g} | | | | | | | | | | |
| Calcium | 0.15 | 0.176 | 0.15 | 0.15 | 0.30 | 0.176 | — | 0.1-0.15 | — | — |
| Chloride | | | | | | | | | | |
| DTPA ^{h} | 0.017 | 0.017 | 0.017 | 0.017 | 0.007 | 0.007 | 0.20 | — | 0.002 | 0.002 |
| Preservative | 5 | 5 | 5 | 5 | 5 | 5 | — | 250 ^{j} | 5 | 5 |
| (ppm) ^{i, j} | | | | | | | | | | |
| Antifoam^{k} | 0.015 | 0.018 | 0.015 | 0.015 | 0.015 | 0.015 | — | — | 0.015 | 0.015 |
| Dye | 40 | 40 | 40 | 40 | 40 | 40 | 11 | 30-300 | 30 | 30 |
| (ppm) | | | | | | | | | | |
| Ammonium | 0.100 | 0.118 | 0.100 | 0.100 | 0.115 | 0.115 | — | — | — | — |
| Chloride | | | | | | | | | | |
| HCl | 0.012 | 0.014 | 0.012 | 0.012 | 0.028 | 0.028 | 0.016 | 0.025 | 0.011 | 0.011 |
| Structurant^{l} | 0.01 | 0.01 | 0.01 | 0.01 | 0.01 | 0.01 | 0.01 | 0.01 | 0.01 | 0.01 |
| Neat | 0.8 | 0.7 | 0.9 | 0.5 | 1.2 | 0.5 | 1.1 | 0.6 | 1.0 | 0.9 |
| Unencapsulated | | | | | | | | | | |
| Perfume | | | | | | | | | | |
| Deionized | Balance | Balance | Balance | Balance | Balance | Balance | Balance | Balance | Balance | Balance |
| Water | | | | | | | | | | |

^{a} N,N-di(tallowoyloxyethyl)-N,N-dimethylammonium chloride.
^{f} Copolymer of ethylene oxide and terephthalate having the formula described in U.S. Pat. No. 5,574,179 at col. 15, lines 1-5, wherein each X is methyl, each n is 40, u is 4, each R1 is essentially 1,4-phenylene moieties, each R2 is essentially ethylene, 1,2-propylene moieties, or mixtures thereof.
^{g} SE39 from Wacker
^{h} Diethylenetriaminepentaacetic acid.
ⁱ KATHON CG available from Rohm and Haas Co. “PPM” is “parts per million.”
^{j} Gluteraldehyde
^{k}Silicone antifoam agent available from Dow Corning Corp. under the trade name DC2310.
^{l}Hydrophobically-modified ethoxylated urethane available from Rohm and Haas under the tradename Aculyn ™ 44.
*Suitable microcapsules provided in Examples 3. (Percent active relates to the core content of the microcapsule)

### Example 14—Dry Laundry Formulations

Non-limiting examples of product formulations containing purified perfume microcapsules of the aforementioned examples are summarized in the following table.

TABLE 14
Dry Laundry Formulations (Examples 14A-14G)

| | % w/w granular laundry detergent composition | | | | | | |
|---|---|---|---|---|---|---|---|
| | Example | | | | | | |
| Ingredient | 14A | 14B | 14C | 14D | 14E | 14F | 14G |
| Brightener | 0.1 | 0.1 | 0.1 | 0.2 | 0.1 | 0.2 | 0.1 |
| Soap | 0.6 | 0.6 | 0.6 | 0.6 | 0.6 | 0.6 | 0.6 |
| Ethylenediamine disuccinic acid | 0.1 | 0.1 | 0.1 | 0.1 | 0.1 | 0.1 | 0.1 |
| Acrylate/maleate copolymer | 1.5 | 1.5 | 1.5 | 1.5 | 1.5 | 1.5 | 1.5 |
| Hydroxyethane di(methylene | 0.4 | 0.4 | 0.4 | 0.4 | 0.4 | 0.4 | 0.4 |
| phosphonic acid) | | | | | | | |
| Mono-C₁₂₋₁₄ alkyl, di-methyl, | 0.5 | 0.5 | 0.5 | 0.5 | 0.5 | 0.5 | 0.5 |
| mono-hydroyethyl quaternary | | | | | | | |
| ammonium chloride | | | | | | | |
| Linear alkyl benzene | 0.1 | 0.1 | 0.2 | 0.1 | 0.1 | 0.2 | 0.1 |
| Linear alkyl benzene sulphonate | 10.3 | 10.1 | 19.9 | 14.7 | 10.3 | 17 | 10.5 |
| Magnesium sulphate | 0.4 | 0.4 | 0.4 | 0.4 | 0.4 | 0.4 | 0.4 |
| Sodium carbonate | 19.5 | 19.2 | 10.1 | 18.5 | 29.9 | 10.1 | 16.8 |
| Sodium sulphate | QS | QS | QS | QS | QS | QS | QS |
| Sodium Chloride | 0.1 | 0.1 | 0.1 | 0.1 | 0.1 | 0.1 | 0.1 |
| Zeolite | 9.6 | 9.4 | 8.1 | 18 | 10 | 13.2 | 17.3 |
| Photobleach particle | 0.1 | 0.1 | 0.2 | 0.1 | 0.2 | 0.1 | 0.2 |
| Blue and red carbonate speckles | 1.8 | 1.8 | 1.8 | 1.8 | 1.8 | 1.8 | 1.8 |
| Ethoxylated Alcohol AE7 | 1 | 1 | 1 | 1 | 1 | 1 | 1 |
| Tetraacetyl ethylene diamine | 0.9 | 0.9 | 0.9 | 0.9 | 0.9 | 0.9 | 0.9 |
| agglomerate (92 wt. % active) | | | | | | | |
| Citric acid | 1.4 | 1.4 | 1.4 | 1.4 | 1.4 | 1.4 | 1.4 |
| Polyethylene oxide | 0.2 | 0.2 | 0.2 | 0.2 | 0.2 | 0.2 | 0.2 |
| Enzymes e.g. Protease (84 mg/g | 0.2 | 0.3 | 0.2 | 0.1 | 0.2 | 0.1 | 0.2 |
| active), Amylase (22 mg/g active) | | | | | | | |
| Suds suppressor agglomerate | 0.2 | 0.2 | 0.2 | 0.2 | 0.2 | 0.2 | 0.2 |
| (12.4 wt. % active) | | | | | | | |
| Sodium percarbonate (having | 7.2 | 7.1 | 4.9 | 5.4 | 6.9 | 19.3 | 13.1 |
| from 12% to 15% active AvOx) | | | | | | | |
| Perfume oil | 0.5 | 0.5 | 0.5 | 0.5 | 0.5 | 0.5 | 0.5 |
| Solid perfume particles | 0.4 | 0 | 0.4 | 0.4 | 0.4 | 0.4 | 0.6 |
| Perfume microcapsules | 1.3 | 2.4 | 1 | 1.3 | 1.3 | 1.3 | 0.7 |
| (Example 3) | | | | | | | |
| Water | 1.4 | 1.4 | 1.4 | 1.4 | 1.4 | 1.4 | 1.4 |
| Misc | 0.1 | 0.1 | 0.1 | 0.1 | 0.1 | 0.1 | 0.1 |
| Total Parts | 100 | 100 | 100 | 100 | 100 | 100 | 100 |

QS—as used herein indicates that this material is used to bring the total to 100%.

### Example 15—Liquid Laundry Formulations (HDLs)

Non-limiting examples of product formulations containing purified perfume microcapsules of the aforementioned examples are summarized in Tables 15.1, 15.2 and 15.3 below.

TABLE 15.1
Liquid Laundry Formulations (HDLs)

| | Example | | | | | |
|---|---|---|---|---|---|---|
| Ingredient | 15A | 15B | 15C | 15D | 15E | 15F |
| Alkyl Ether Sulphate | 0.00 | 0.50 | 12.0 | 12.0 | 6.0 | 7.0 |
| Dodecyl Benzene | 8.0 | 8.0 | 1.0 | 1.0 | 2.0 | 3.0 |
| Sulphonic Acid | | | | | | |
| Ethoxylated Alcohol | 8.0 | 6.0 | 5.0 | 7.0 | 5.0 | 3.0 |
| Citric Acid | 5.0 | 3.0 | 3.0 | 5.0 | 2.0 | 3.0 |
| Fatty Acid | 3.0 | 5.0 | 5.0 | 3.0 | 6.0 | 5.0 |
| Ethoxysulfated | 1.9 | 1.2 | 1.5 | 2.0 | 1.0 | 1.0 |
| hexamethylene diamine | | | | | | |
| quaternized | | | | | | |
| Diethylene triamine | 0.3 | 0.2 | 0.2 | 0.3 | 0.1 | 0.2 |
| pentamethylene phosphonic | | | | | | |
| acid | | | | | | |
| Enzymes | 1.20 | 0.80 | 0 | 1.2 | 0 | 0.8 |
| Brightener (disulphonated | 0.14 | 0.09 | 0 | 0.14 | 0.01 | 0.09 |
| diamino stilbene based | | | | | | |
| FWA) | | | | | | |
| Cationic hydroxyethyl | 0 | 0 | 0.10 | 0 | 0.200 | 0.30 |
| cellulose | | | | | | |
| Poly(acrylamide-co- | 0 | 0 | 0 | 0.50 | 0.10 | 0 |
| diallyldimethylammonium | | | | | | |
| chloride) | | | | | | |
| Hydrogenated Castor Oil | 0.50 | 0.44 | 0.2 | 0.2 | 0.3 | 0.3 |
| Structurant | | | | | | |
| Boric acid | 2.4 | 1.5 | 1.0 | 2.4 | 1.0 | 1.5 |
| Ethanol | 0.50 | 1.0 | 2.0 | 2.0 | 1.0 | 1.0 |
| 1,2 propanediol | 2.0 | 3.0 | 1.0 | 1.0 | 0.01 | 0.01 |
| Diethyleneglycol (DEG) | 1.6 | 0 | 0 | 0 | 0 | 0 |
| 2,3-Methyl-1,3- | 1.0 | 1.0 | 0 | 0 | 0 | 0 |
| propanediol (M pdiol) | | | | | | |
| Mono Ethanol Amine | 1.0 | 0.5 | 0 | 0 | 0 | 0 |
| NaOH Sufficient To | pH 8 | pH 8 | pH 8 | pH 8 | pH 8 | pH 8 |
| Provide Formulation | | | | | | |
| pH of: | | | | | | |
| Sodium Cumene | 2.00 | 0 | 0 | 0 | 0 | 0 |
| Sulphonate (NaCS) | | | | | | |
| Perfume | 0.7 | 0.5 | 0.8 | 0.8 | 0.6 | 0.6 |
| Polyethyleneimine | 0.01 | 0.10 | 0.00 | 0.10 | 0.20 | 0.05 |
| Perfume Microcapsules | 1.00 | 5.00 | 1.00 | 2.00 | 0.10 | 0.80 |
| of Example 3 | | | | | | |
| Water | Balance | Balance | Balance | Balance | Balance | Balance |
| | to | to | to | to | to | to |
| | 100% | 100% | 100% | 100% | 100% | 100% |

TABLE 15.2
Liquid Laundry Detergent Formulations

| | Example | | | |
|---|---|---|---|---|
| Ingredient | 15G | 15H | 15I | 15J |
| C14-C15 alkyl poly ethoxylate (8) | 6.25 | 4.00 | 6.25 | 6.25 |
| C12-C14 alkyl poly ethoxylate (7) | 0.40 | 0.30 | 0.40 | 0.40 |
| C12-C14 alkyl poly ethoxylate (3) | 10.60 | 6.78 | 10.60 | 10.60 |
| sulfate Na salt | | | | |
| Linear Alkylbenzene sulfonate acid | 0.19 | 1.16 | 0.79 | 0.79 |
| Citric Acid | 3.75 | 2.40 | 3.75 | 3.75 |
| C12-C18 Fatty Acid | 4.00 | 2.56 | 7.02 | 7.02 |
| Enzymes | 0.60 | 0.4 | 0.60 | 0.60 |
| Boric Acid | 2.4 | 1.5 | 1.25 | 1.25 |
| Trans-sulphated ethoxylated | 1.11 | 0.71 | 1.11 | 1.11 |
| hexamethylene diamine quat | | | | |
| Diethylene triamine penta | 0.17 | 0.11 | 0.17 | 0.17 |
| methylene phosphonic acid | | | | |
| Fluorescent brightener | 0.09 | 0.06 | 0.14 | 0.14 |
| Hydrogenated Castor Oil | 0.05 | 0.300 | 0.20 | 0.20 |
| Ethanol | 2.50 | 1.00 | 2.50 | 2.50 |
| 1,2 propanediol | 1.14 | 0.7 | 1.14 | 1.14 |
| Sodium hydroxide | 3.8 | 2.6 | 4.60 | 4.60 |
| Mono Ethanol Amine | 0.8 | 0.5 | | |
| Na Cumene Sulphonate | | | 1.0 | |
| Dye | 0.002 | 0.002 | 0.002 | 0.002 |
| Opacifier (Styrene Acrylate based) | 0.1 | | | |
| Bentonite Softening Clay | | 1.0 | | |
| Polyquaternium 10 - Cationic | 1.0 | | 1.0 | 1.0 |
| hydroxyl ethyl cellulose | | | | |
| PP-5495 (silicone ex Dow Corning | | 1.0 | | |
| Corporation, Midland, MI) | | | | |
| DC 1664 (silicone ex Dow Corning | | | 1.0 | |
| Corporation, Midland, MI) | | | | |
| Perfume micro capsules (expressed | 0.8 | 0.5 | 1.0 | 0.7 |
| as perfume oil) of Example 3 | | | | |
| Perfume | 0.7 | 0.55 | 1.00 | 1.00 |
| Poly Ethylene Imine MW 25000 | 0.1 | | | |
| Water | Up | Up | Up | Up |
| | to 100 | to 100 | to 100 | to 100 |

TABLE 15.3
Liquid Laundry Detergent Formulations.

| | Example | | |
|---|---|---|---|
| Ingredient | 15K | 15L | 15M |
| C14-C15 alkyl poly ethoxylate (8) | 3.7 | | 20.7 |
| C12-C14 alkyl poly ethoxylate (7) | | 16.7 | |
| C12-C14 alkyl poly ethoxylate (3) | 17.8 | | 5.5 |
| sulfate Na salt | | | |
| Linear Alkylbenzene sulfonate acid | 12.5 | 22.9 | 13.5 |
| Citric Acid | 3.9 | | 1.7 |
| C12-C18 Fatty Acid | 11.1 | 18 | 5.1 |
| Enzymes | 3 | 1.2 | 3 |
| Boric Acid | 0.5 | | 0.5 |
| Trans-sulphated ethoxylated | 3.25 | | 1.2 |
| hexamethylene diamine quat | | | |
| PEI 600 EO20 | 1.25 | | 1.2 |
| Diethylene triamine penta methylene | 1.6 | | 0.85 |
| phosphonic acid or HEDP | | | |
| Fluorescent brightener | 0.2 | 0.3 | 0.14 |
| Hydrogenated Castor Oil | | 0.2 | |
| 1,2 propanediol | 4.3 | 20.3 | 11.7 |
| Sodium hydroxide | | 1.0 | 3.9 |
| Mono Ethanol Amine | 9.8 | 6.8 | 3.1 |
| Dye | Present | Present | Present |
| PDMS | | 2.15 | |
| Potassium sulphite | | 0.2 | |
| Perfume micro capsules (expressed as | 1.6 | 1.5 | 1.4 |
| perfume oil) of Examples 3 | | | |
| Perfume | 1.2 | 1.6 | 1.0 |
| Form. Phenyl Boronic Acid | | | Present |
| Water** | Up | Up | Up |
| | to 100 | to 100 | to 100 |

**Low water liquid detergent in Polyvinylalcohol unidose/sachet

### Example 16—Liquid and Gel Detergents

Non-limiting examples of product formulations containing purified perfume microcapsules of the aforementioned examples are summarized in Table 16 below.

TABLE 16
Liquid and Gel Detergent Formulations (% by Weight)

| | Example | | |
|---|---|---|---|
| Ingredient | 16A | 16B | 16C |
| Alkylbenzenesulfonic acid | 17.2 | 12.2 | 23 |
| C12-14 alcohol 7-ethoxylate | 8.6 | 0.4 | 19.5 |
| C14-15 alcohol 8-ethoxylate | — | 9.6 | — |
| C12-14 alcohol 3-ethoxylate sulphate, | 8.6 | — | — |
| Na salt | | | |
| C8-10 Alkylamidopropyldimethyl amine | — | — | 0.9 |
| Citric acid | 2.9 | 4.0 | — |
| C12-18 fatty acid | 12.7 | 4.0 | 17.3 |
| Enzymes | 3.5 | 1.1 | 1.4 |
| Ethoxylated polyimine | 1.4 | — | 1.6 |
| Ethoxylated polyimine polymer, quaternized | 3.7 | 1.8 | 1.6 |
| and sulphated | | | |
| Hydroxyethane diphosphonic acids (HEDP) | 1.4 | — | — |
| Pentamethylene triamine pentaphosphonic | — | 0.3 | — |
| acid | | | |
| Catechol 2,5 disulfonate, Na salt | 0.9 | — | — |
| Fluorescent whitening agent | 0.3 | 0.15 | 0.3 |
| 1,2 propandiol | 3.5 | 3.3 | 22 |
| Ethanol | — | 1.4 | — |
| Diethylene glycol | — | 1.6 | — |
| 1-ethoxypentanol | 0.9 | — | — |
| Sodium cumene sulfonate | | 0.5 | — |
| Monoethanolamine (MEA) | 10.2 | 0.8 | 8.0 |
| MEA borate | 0.5 | 2.4 | — |
| Sodium hydroxide | — | 4.6 | — |
| Perfume | 1.6 | 0.7 | 1.5 |
| Perfume microcapsules of Example 3 | 1.1 | 1.2 | 0.9 |
| Water | 22.1 | 50.8 | 2.9 |
| Perfume, dyes, miscellaneous minors | Balance | Balance | Balance |
| Undiluted viscosity (Vₙ) at 20 s⁻¹, cps | 2700 | 400 | 300 |

### Example 17—Liquid Unit Dose

The following are examples of unit dosage forms wherein the liquid composition is enclosed within a PVA film. The preferred film used in the present examples is Monosol M8630 76 μm thickness.

TABLE 17
Unit Dose Laundry Cleaner

| | Example | | | | | | | |
|---|---|---|---|---|---|---|---|---|
| | 17A | | | 17B | | 17C | | |
| | 3 compartments | | | 2 compartments | | 3 compartments | | |
| | Compartment # | | | | | | | |
| | 42 | 43 | 44 | 45 | 46 | 47 | 48 | 49 |
| | Dosage (g) | | | | | | | |
| | 34.0 | 3.5 | 3.5 | 30.0 | 5.0 | 25.0 | 1.5 | 4.0 |
| Ingredients | Weight % | | | | | | | |
| Alkylbenzene sulfonic acid | 20.0 | 20.0 | 20.0 | 10.0 | 20.0 | 20.0 | 25 | 30 |
| Alkyl sulfate | | | | 2.0 | | | | |
| C₁₂₋₁₄ alkyl 7-ethoxylate | 17.0 | 17.0 | 17.0 | | 17.0 | 17.0 | 15 | 10 |
| C₁₂₋₁₄ alkyl ethoxy 3 sulfate | 7.5 | 7.5 | 7.5 | | | 7.5 | 7.5 | |
| Citric acid | 0.5 | | 2.0 | 1.0 | | | | 2.0 |
| Zeolite A | | | | 10.0 | | | | |
| C₁₂₋₁₈ Fatty acid | 13.0 | 13.0 | 13.0 | | 18.0 | 18.0 | 10 | 15 |
| Sodium citrate | | | | 4.0 | 2.5 | | | |
| Enzymes | 0-3 | 0-3 | 0-3 | 0-3 | | 0-3 | 0-3 | 0-3 |
| Sodium Percarbonate | | | | 11.0 | | | | |
| TAED | | | | 4.0 | | | | |
| Polycarboxylate | | | | 1.0 | | | | |
| Ethoxylated | 2.2 | 2.2 | 2.2 | | | | | |
| Polyethylenimine¹ | | | | | | | | |
| Hydroxyethane | 0.6 | 0.6 | 0.6 | 0.5 | | | 2.2 | |
| diphosphonic acid | | | | | | | | |
| Ethylene diamine | | | | | | 0.4 | | |
| tetra(methylene phosphonic) | | | | | | | | |
| acid | | | | | | | | |
| Brightener | 0.2 | 0.2 | 0.2 | 0.3 | | 0.3 | | |
| Microcapsules of | 0.4 | 1.2 | 1.5 | 1.3 | 1.3 | 0.4 | 0.12 | 0.2 |
| examples 3 | | | | | | | | |
| Water | 9 | 8.5 | 10 | 5 | 11 | 10 | 10 | 9 |
| CaCl2 | | | | | | | 0.01 | |
| Perfume | 1.7 | 1.7 | | 0.6 | | 1.5 | 0.5 | |
| Minors (antioxidant, sulfite, | 2.0 | 2.0 | 2.0 | 4.0 | 1.5 | 2.2 | 2.2 | 2.0 |
| aesthetics, . . . ) | | | | | | | | |

| | |
|---|---|
| Buffers (sodium | To pH 8.0 for liquids |
| carbonate, | To RA > 5.0 for powders |
| monoethanolamine) ² | |
| Solvents (1,2 propanediol, | To 100 p |
| ethanol), sodium sulfate | |

¹Polyethylenimine (MW = 600) with 20 ethoxylate groups per —NH.
² RA = Reserve Alkalinity (g NaOH/dose)

### Example 18—Scent Booster

Microcapsules can also be incorporated into a laundry scent booster. Exemplary scent booster formulations are presented in Table 18.1, 18.2, and 18.3.

TABLE 18.1

| Material | Parts |
|---|---|
| Urea beads | 86 |
| Bentonite | 3 |
| Perfume oil | 3 |
| Spray Dried Microcapsules of Example 3 | 8 |

Powders are mixed together using a low shear powder blender.

TABLE 18.2

| Material | Parts |
|---|---|
| Sodium Chloride powder | 95 |
| Spray Dried Microcapsules of Example 3 | 5 |

Powders are mixed together using a low shear powder blender.

TABLE 18.3

| Material | Parts |
|---|---|
| Polyethylene Glycol 8000 | 87 |
| Dipropylene Glycol | 1.8 |
| Perfume oil | 7.0 |
| Spray Dried Microcapsules of Example 3 | 3.8 |

Polyethylene glycol 8000 is melted at 75° C. The dipropylene glycol and perfume oil are added and mixed to yield a homogeneous mixture. The spray dried microcapsule powder is incorporated into the melt at 60-65° C. and subsequently extruded into pellets or processed using a steel belt to make pastilles. Such pastille processing equipment is available from SBS Steel Belt Systems.

### ADDITIONAL NUMBERED EMBODIMENTS OF THE INVENTION

1. A composition comprising at least one of:
(a) an amino acid isocyanate (AAI) adduct, which has a structure represented by Formula I:

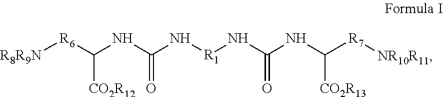

and salts thereof, and
(b) an amino acid epoxide (AAE) adduct, which has a structure represented by Formula II:

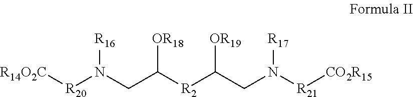

and salts thereof, where
R₁ is a moiety of a monomer or a prepolymer comprising two-NCO functional groups wherein the NCO functional groups have reacted to form urea linkages;
R₂ is a moiety of a monomer or a prepolymer comprising two glycidyl ether epoxide functional groups wherein the epoxide groups have reacted to form amino alcohol groups;
R₆, R₇, R₂₀ and R₂₁ are each independently (CH₂)ₙ, wherein n is 1-6;
R₈, R₉, R₁₀ and R₁₁ are each independently hydrogen or CH₂X;
R₁₂ and R₁₃ are each independently hydrogen, an alkali metal cation, or X;
R₁₄ and R₁₅ are each independently hydrogen, an alkali metal cation, or X;
R₁₆ and R₁₇ are each independently hydrogen or X;
R₁₈ and R₁₉ are each independently hydrogen, SiO₃ or X; and
each occurrence of X is independently selected from the group consisting of hydrogen and a substituent.
2. The composition of embodiment 1, comprising the AAI adduct, wherein:
R₆ and R₇ are each (CH₂)₄;
R₈, R₉, R₁₀ and R₁₁ are each hydrogen; and
R₁₂ and R₁₃ are each independently hydrogen or an alkali metal cation.
3. The composition of embodiment 1 or 2, wherein the AAI adduct is a reaction product of: (a) an amino acid or any modified form of amino acid comprising: (i) two reactive primary amines groups; (ii) two reactive secondary amines groups; or (iii) one reactive primary amine group and one reactive secondary amine group; and (b) a diisocyanate monomer or prepolymer comprising two reactive isocyanate groups, wherein the diisocyanate monomer or prepolymer is used at a stoichiometric molar ratio of isocyanate:amine of 1:2.
4. The composition of embodiment 3, wherein the amino acid comprises at least one member selected from the group consisting of lysine, arginine, ornithine, 2,4-diaminobutyric acid and lanthionine.
5. The composition of embodiment 3 or 4, wherein the diisocyanate monomer or prepolymer comprises at least one member selected from the group consisting of isophorone diisocyanate, toluene diisocyanate, hexamethylene diisocyanate, xylene diisocyanate, pentamethylene diisocyanate, 4,4′-methylene diphenyl diisocyanate and 4,4′-methylene dicyclohexyl diisocyanate.
6. The composition of any one of embodiments 1-5, comprising the AAE adduct, wherein:
R₁₄ and R₁₅ are each independently hydrogen or an alkali metal cation;
R₁₆ and R₁₇ are each hydrogen;
R₁₈ and R₁₉ are each independently hydrogen or SiO₃; and
R₂₀ and R₂₁ are each (CH₂)₂.
7. The composition of any one of embodiments 1-6, wherein the AAE adduct is a reaction product of: (a) an amino acid comprising at least one reactive amine which is chemically bonded to at least one carbon that is separated from a carbonyl carbon of a carboxylic acid of the amino acid by at least one carbon, and is one primary amine or two secondary amines; and (b) a diepoxy monomer or prepolymer having two glycidyl ether-based epoxide groups, wherein the diepoxy monomer or prepolymer is used at an equivalent ratio of epoxy:amine of 1:2.
8. The composition of embodiment 7, wherein the amino acid is at least one of beta-alanine, beta-aminobutyric acid and beta-leucine.
9. The composition of embodiment 7, wherein the diepoxy monomer or prepolymer is a member selected from the group consisting of resorcinol diglycidyl ether, bisphenol-A diglycidyl ether, bisphenol-F diglycidyl ether and hydrogenated bisphenol-A diglycidyl ether.
10. The composition of any one of embodiments 1-9, which is a polymer comprising more than one of the AAI adduct or more than one of the AAE adduct.
11. The composition of any one of embodiments 1-9, which is a copolymer comprising at least one of the AAI adduct and at least one of the AAE adduct.
12. A controlled release particle comprising:
a core that comprises at least one hydrophobic active ingredient, optionally a sugar alcohol, and optionally a plasticizer; and
a shell that comprises a reaction product of (a) at least one isocyanate resin and optionally an epoxy resin with (b) optionally a hydrolyzed organofunctional silane, at least one inorganic solid particle capable of coupling with a carboxylic acid group, and at least one of the AAI adduct and the AAE adduct of any one of embodiments 1-11.
13. The controlled release particle of embodiment 12, wherein the at least one hydrophobic active ingredient comprises at least one member selected from the group consisting of a flavorant, a fragrance, a chromogen, a dye, an essential oil, a sweetener, an oil, a pigment, an active pharmaceutical ingredient, a moldicide, a herbicide, a fertilizer, a phase change material, an adhesive, a vitamin oil, a vegetable oil, a triglyceride and a hydrocarbon.
14. The controlled release particle of embodiment 12 or 13, wherein the core comprises the sugar alcohol, which is an alcohol of a monosaccharide, disaccharide, trisaccharide, tetra-saccharide or penta-saccharide.
15. The controlled release particle of any one of embodiments 12-14, wherein the core comprises the plasticizer, which is a member selected from the group consisting of methyl esters of rosin, polyazelate esters, di-fatty acid esters, citrate esters, polyadipate esters and polyester resins consisting of inner and intra-esters of polyhydroxy carboxylic acids.
16. The controlled release particle of any one of embodiments 12-15, wherein the at least one isocyanate resin comprises a material selected from the group consisting of an aliphatic isocyanate, an aromatic isocyanate, a polymeric isocyanate, a cyclic isocyanate, a hydrophilic isocyanate, a hydrophobic isocyanate, an isocyanurate, a waterborne isocyanate and a urethane acrylate containing isocyanate functionalities.
17. The controlled release particle of any one of embodiments 12-16, wherein the epoxy resin is reacted to provide the reaction product and comprises a material selected from the group consisting of an epoxidized unsaturated oil, an epoxidized vegetable oil, an epoxidized alcohol, an epoxidized silane, an epoxidized polysaccharide, a trimethylol propane triglycidyl ether, tetraglycidy ether sorbitol, multi-glycidyl ether phenol novolac, a resin containing acrylate and epoxy functional groups, a diepoxide of a cycloapliphatic alcohol, a hydrogenated Bisphenol A, and a resorcinol/bisphenol F resin with polyfunctional epoxide resin blend.
18. The controlled release particle of any one of embodiments 12-17, wherein the organofunctional silane is reacted to provide the reaction product and comprises a material selected from the group consisting of hydrolyzed alkoxylated silanes, hydrolyzed trialkoxy silanes, hydrolyzed tetraalkoxyated silanes and hydrolyzed glycidyl ether silanes.
19. The controlled release particle of any one of embodiments 12-18, wherein the at least one inorganic solid particle comprises a material selected from the group consisting of sodium silicate, sodium metasilicate, organically modified clay, water insoluble clay, minerals, talc, calcium carbonate, bentonite, calcium chloride, magnesium sulfate, hydroxyapatite, calcium phosphate, kaolin, montmorrilonite and amine-modified kaolin.
20. The controlled release particle of any one of embodiments 12-19, having a diameter of 1-150 μm.
21. A consumer product comprising a plurality of the controlled release particles of any one of embodiments 12-20, wherein the consumer product is selected from the group consisting of a powdered food product, a fluid food product, a powdered nutritional supplement, a fluid nutritional supplement, a fluid fabric enhancer, a solid fabric enhancer, a fluid shampoo, a solid shampoo, a hair conditioner, a body wash, a solid antiperspirant, a fluid antiperspirant, a solid deodorant, a fluid deodorant, a fluid detergent, a solid detergent, a fluid hard surface cleaner, a solid hard surface cleaner, a fluid fabric refresher spray, a diaper, an air freshening product, a nutraceutical supplement, a controlled release fertilizer, a controlled release insecticide, a controlled release dye and a unit dose detergent further comprising a detergent and a water soluble outer film.
22. A method of making the composition of any one of embodiments 1-11 consisting of the AAI adduct, said method comprising the steps of:
(a) preparing an aqueous solution of an amino acid hydrochloride, wherein amino acids thereof comprise amines and carboxylic acids;
(b) adding an equivalent amount of an aqueous solution of sodium hydroxide to the aqueous solution of the amino acid hydrochloride to provide a modified solution;
(c) adding a diisocyanate dropwise into the modified solution kept at room temperature to 40° C.;
(d) further reacting the diisocyanate at an elevated temperature above 40° C. to complete consumption of isocyanates of the diisocyanate by the amines;
(e) adding a second equivalent amount of aqueous solution of sodium hydroxide to neutralize the carboxylic acids; and
(f) optionally filtering a product of step (e).
23. A method of making the composition of any one of embodiments 1-11 consisting of the AAE adduct, said method comprising the steps of:
(a) preparing an aqueous solution of amino acids;
(b) adding an equivalent amount of an aqueous solution of sodium hydroxide to the aqueous solution of amino acids to provide a modified solution;
(c) adding a reactive glycidyl ether based diepoxy dropwise into the modified solution kept at a temperature of 20-75° C.;
(d) further reacting epoxides of the diepoxy with amines of the amino acids at an elevated temperature of 75-85° C. to complete consumption of the epoxides by the amines; and
(e) optionally filtering the product.
24. A method of making the controlled release particles of any one of embodiments 12-20, said method comprising the steps of:
(a) preparing a core material by mixing the at least one hydrophobic active ingredient with the at least one isocyanate resin, optionally an epoxy resin, optionally the sugar alcohol, and optionally the plasticizer to provide a core material phase which is a solution or suspension;
(b) providing an emulsifier composition which is a homogeneous aqueous solution or aqueous dispersion of at least one emulsifier;
(c) optionally providing an amine functional low molecular weight protein or peptide;
(d) adding the core material phase into the emulsifier composition to provide an oil-in-water emulsion at room temperature to 65° C.;
(e) adding an aqueous solution of the isocyanate adduct of Formula I and/or Formula II into the oil-in-water emulsion;
(f) heating the oil-in-water emulsion from step (d) to a temperature of 35° C. to 65° C. to form a shell via an interfacial polymerization reaction;
(g) optionally adding tetrahydroxy orthosiloxane or orthosilicic acid to the oil-in-water emulsion from step (e), followed by removal of ethanol;
(h) further adding to the oil-in-water emulsion from step (f) an aqueous solution of an inorganic solid particles salt comprising at least a divalent cation capable of coupling with carboxylic acids, to provide a suspension of the controlled release particles;
(i) optionally, adding suspension or thickening agents to stabilize the microcapsules homogeneously in the slurry;
(j) optionally filtering the suspension from step (h) to form a free-flowing semi-dry powder;
(k) optionally drying further the semi-dry powder in an oven to yield a dry powder; and
(l) optionally post-curing the dry powder at an elevated temperature of 100° C. to 150° C. for 30 minutes to 60 minutes.

While the invention has been described in detail and with reference to specific examples thereof, it will be apparent to one skilled in the art that various changes and modifications can be made therein without departing from the spirit and scope thereof.

What is claimed is:

1. A composition comprising at least one of:
(a) an amino acid isocyanate (AAI) adduct, which has a structure represented by Formula I: 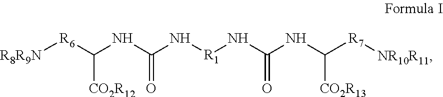
and salts thereof, and
(b) an amino acid epoxide (AAE) adduct, which has a structure represented by Formula II: 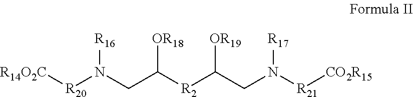
and salts thereof, where
R₁ is a moiety of a monomer or a prepolymer comprising two-NCO functional groups wherein the NCO functional groups have reacted to form urea linkages;
R₂ is a moiety of a monomer or a prepolymer comprising two glycidyl ether epoxide functional groups wherein the epoxide groups have reacted to form amino alcohol groups;
R₆, R₇, R₂₀ and R₂₁ are each independently (CH₂)ₙ, wherein n is 1-6;
R₈, R₉, R₁₀ and Ru are each independently hydrogen or CH₂X;
R₁₂ and R₁₃ are each independently hydrogen, an alkali metal cation, or X;
R₁₄ and R₁₅ are each independently hydrogen, an alkali metal cation, or X;
R₁₆ and R₁₇ are each independently hydrogen or X;
R₁₈ and R₁₉ are each independently hydrogen, SiO₃ or X; and
each occurrence of X is independently selected from the group consisting of hydrogen and a substituent.

2. The composition of claim 1, comprising the AAI adduct, wherein:
R₆ and R₇ are each (CH₂)₄;
R₈, R₉, R₁₀ and R₁₁ are each hydrogen; and
R₁₂ and R₁₃ are each independently hydrogen or an alkali metal cation.

3. The composition of claim 2, wherein the AAI adduct is a reaction product of: (a) an amino acid or any modified form of amino acid comprising: (i) two reactive primary amines groups; (ii) two reactive secondary amines groups; or (iii) one reactive primary amine group and one reactive secondary amine group; and (b) a diisocyanate monomer or prepolymer comprising two reactive isocyanate groups, wherein the diisocyanate monomer or prepolymer is used at a stoichiometric molar ratio of isocyanate:amine of 1:2.

4. The composition of claim 3, wherein the amino acid comprises at least one member selected from the group consisting of lysine, arginine, ornithine, 2,4-diaminobutyric acid and lanthionine.

5. The composition of claim 3, wherein the diisocyanate monomer or prepolymer comprises at least one member selected from the group consisting of isophorone diisocyanate, toluene diisocyanate, hexamethylene diisocyanate, xylene diisocyanate, pentamethylene diisocyanate, 4,4′-methylene diphenyl diisocyanate and 4,4′-methylene dicyclohexyl diisocyanate.

6. The composition of claim 1, comprising the AAE adduct, wherein:
R₁₄ and R₁₅ are each independently hydrogen or an alkali metal cation;
R₁₆ and R₁₇ are each hydrogen;
R₁₈ and R₁₉ are each independently hydrogen or SiO₃; and
R₂₀ and R₂₁ are each (CH₂)₂.

7. The composition of claim 6, wherein the AAE adduct is a reaction product of: (a) an amino acid comprising at least one reactive amine which is chemically bonded to at least one carbon that is separated from a carbonyl carbon of a carboxylic acid of the amino acid by at least one carbon, and is one primary amine or two secondary amines; and (b) a diepoxy monomer or prepolymer having two glycidyl ether-based epoxide groups, wherein the diepoxy monomer or prepolymer is used at an equivalent ratio of epoxy:amine of 1:2.

8. The composition of claim 7, wherein the amino acid is at least one of beta-alanine, beta-aminobutyric acid and beta-leucine.

9. The composition of claim 7, wherein the diepoxy monomer or prepolymer is a member selected from the group consisting of resorcinol diglycidyl ether, bisphenol-A diglycidyl ether, bisphenol-F diglycidyl ether and hydrogenated bisphenol-A diglycidyl ether.

10. The composition of claim 1, which is a polymer comprising more than one of the AAI adduct or more than one of the AAE adduct.

11. The composition of claim 1, which is a copolymer comprising at least one of the AAI adduct and at least one of the AAE adduct.

12. A controlled release particle comprising:
a core that comprises at least one hydrophobic active ingredient, optionally a sugar alcohol, and optionally a plasticizer; and
a shell that comprises a reaction product of (a) at least one isocyanate resin and optionally an epoxy resin with (b) optionally a hydrolyzed organofunctional silane, at least one inorganic solid particle capable of coupling with a carboxylic acid group, and at least one of the AAI adduct and the AAE adduct of claim 1.

13. The controlled release particle of claim 12, wherein the at least one hydrophobic active ingredient comprises at least one member selected from the group consisting of a flavorant, a fragrance, a chromogen, a dye, an essential oil, a sweetener, an oil, a pigment, an active pharmaceutical ingredient, a moldicide, a herbicide, a fertilizer, a phase change material, an adhesive, a vitamin oil, a vegetable oil, a triglyceride and a hydrocarbon.

14. The controlled release particle of claim 12, wherein the core comprises the sugar alcohol, which is an alcohol of a monosaccharide, disaccharide, trisaccharide, tetra-saccharide or penta-saccharide.

15. The controlled release particle of claim 12, wherein the core comprises the plasticizer, which is a member selected from the group consisting of methyl esters of rosin, polyazelate esters, di-fatty acid esters, citrate esters, polyadipate esters and polyester resins consisting of inner and intra-esters of polyhydroxy carboxylic acids.

16. The controlled release particle of claim 12, wherein the at least one isocyanate resin comprises a material selected from the group consisting of an aliphatic isocyanate, an aromatic isocyanate, a polymeric isocyanate, a cyclic isocyanate, a hydrophilic isocyanate, a hydrophobic isocyanate, an isocyanurate, a waterborne isocyanate and a urethane acrylate containing isocyanate functionalities.

17. The controlled release particle of claim 12, wherein the epoxy resin is reacted to provide the reaction product and comprises a material selected from the group consisting of an epoxidized unsaturated oil, an epoxidized vegetable oil, an epoxidized alcohol, an epoxidized silane, an epoxidized polysaccharide, a trimethylol propane triglycidyl ether, tetraglycidy ether sorbitol, multi-glycidyl ether phenol novolac, a resin containing acrylate and epoxy functional groups, a diepoxide of a cycloapliphatic alcohol, a hydrogenated Bisphenol A, and a resorcinol/bisphenol F resin with polyfunctional epoxide resin blend.

18. The controlled release particle of claim 12, wherein the organofunctional silane is reacted to provide the reaction product and comprises a material selected from the group consisting of hydrolyzed alkoxylated silanes, hydrolyzed trialkoxy silanes, hydrolyzed tetraalkoxyated silanes and hydrolyzed glycidyl ether silanes.

19. The controlled release particle of claim 12, wherein the at least one inorganic solid particle comprises a material selected from the group consisting of sodium silicate, sodium metasilicate, organically modified clay, water insoluble clay, minerals, talc, calcium carbonate, bentonite, calcium chloride, magnesium sulfate, hydroxyapatite, calcium phosphate, kaolin, montmorrilonite and amine-modified kaolin.

20. The controlled release particle of claim 12, having a diameter of 1-150 μm.

21. A consumer product comprising a plurality of the controlled release particles of claim 12, wherein the consumer product is selected from the group consisting of a powdered food product, a fluid food product, a powdered nutritional supplement, a fluid nutritional supplement, a fluid fabric enhancer, a solid fabric enhancer, a fluid shampoo, a solid shampoo, a hair conditioner, a body wash, a solid antiperspirant, a fluid antiperspirant, a solid deodorant, a fluid deodorant, a fluid detergent, a solid detergent, a fluid hard surface cleaner, a solid hard surface cleaner, a fluid fabric refresher spray, a diaper, an air freshening product, a nutraceutical supplement, a controlled release fertilizer, a controlled release insecticide, a controlled release dye and a unit dose detergent further comprising a detergent and a water soluble outer film.

22. A method of making the composition of claim 1 consisting of the AAI adduct, said method comprising the steps of:
(a) preparing an aqueous solution of an amino acid hydrochloride, wherein amino acids thereof comprise amines and carboxylic acids;
(b) adding an equivalent amount of an aqueous solution of sodium hydroxide to the aqueous solution of the amino acid hydrochloride to provide a modified solution;
(c) adding a diisocyanate dropwise into the modified solution kept at room temperature to 40° C.;
(d) further reacting the diisocyanate at an elevated temperature above 40° C. to complete consumption of isocyanates of the diisocyanate by the amines;
(e) adding a second equivalent amount of aqueous solution of sodium hydroxide to neutralize the carboxylic acids; and
(f) optionally filtering a product of step (e).

23. A method of making the composition of claim 1 consisting of the AAE adduct, said method comprising the steps of:
(a) preparing an aqueous solution of amino acids;
(b) adding an equivalent amount of an aqueous solution of sodium hydroxide to the aqueous solution of amino acids to provide a modified solution;
(c) adding a reactive glycidyl ether based diepoxy dropwise into the modified solution kept at a temperature of 20-75° C.;
(d) further reacting epoxides of the diepoxy with amines of the amino acids at an elevated temperature of 75-85° C. to complete consumption of the epoxides by the amines; and
(e) optionally filtering the product.

24. A method of making the controlled release particles of claim 12, said method comprising the steps of:
(a) preparing a core material by mixing the at least one hydrophobic active ingredient with the at least one isocyanate resin, optionally an epoxy resin, optionally the sugar alcohol, and optionally the plasticizer to provide a core material phase which is a solution or suspension;
(b) providing an emulsifier composition which is a homogeneous aqueous solution or aqueous dispersion of at least one emulsifier;
(c) optionally providing an amine functional low molecular weight protein or peptide;
(d) adding the core material phase into the emulsifier composition to provide an oil-in-water emulsion at room temperature to 65° C.;
(e) adding an aqueous solution of the isocyanate adduct of Formula I and/or Formula II into the oil-in-water emulsion;
(f) heating the oil-in-water emulsion from step (d) to a temperature of 35° C. to 65° C. to form a shell via an interfacial polymerization reaction;
(g) optionally adding tetrahydroxy orthosiloxane or orthosilicic acid to the oil-in-water emulsion from step (e), followed by removal of ethanol;
(h) further adding to the oil-in-water emulsion from step (f) an aqueous solution of an inorganic solid particles salt comprising at least a divalent cation capable of coupling with carboxylic acids, to provide a suspension of the controlled release particles;
(i) optionally, adding suspension or thickening agents to stabilize the microcapsules homogeneously in the slurry;
(j) optionally filtering the suspension from step (h) to form a free-flowing semi-dry powder,
(k) optionally drying further the semi-dry powder in an oven to yield a dry powder; and
(l) optionally post-curing the dry powder at an elevated temperature of 100° C. to 150° C. for 30 minutes to 60 minutes.
